(19) United States
(12) Patent Application Publication
(10) Pub. No.: US 2026/0259228 A1
(43) Pub. Date: Sep. 3, 2026

(54) METHODS OF PROGNOSIS AND TREATMENT FOR HEART FAILURE WITH PRESERVED EJECTION FRACTION USING URINARY PROTEIN LEVELS

(71) Applicants: Bristol Myers Squibb Company, Princeton, NJ (US); The Trustees of the University of Pennsylvania, Philadelphia, PA (US)

(72) Inventors: Lei ZHAO, Princeton, NJ (US); Julio CHIRINOS, Philadelphia, PA (US); Ashok DONGRE, Princeton, NJ (US)

(73) Assignee: The Trustees of the University of Pennsylvania, Philadelphia, PA (US)

(21) Appl. No.: 19/156,259

(22) PCT Filed: Feb. 16, 2024
(86) PCT No.: PCT/US2024/016105

§ 371 Date: Aug. 13, 2025

Related U.S. Application Data
(60) Provisional application No. 63/485,770, filed on Feb. 17, 2023.
(60) Provisional application No. 63/488,158, filed on Mar. 2, 2023.

(51) Int. Cl. G01N 33/68

(52) U.S. Cl. CPC G01N 33/6893; G01N 2800/325; G01N 2800/50; G01N 2800/52

(57) ABSTRACT

Methods of prognosis and treatment of subjects with heart failure with preserved ejection fraction (HFpEF) based on urinary protein levels are described herein. Such methods may include obtaining a urine sample of a subject (e.g., a HFpEF patient), measuring urinary protein levels in the sample, analyzing the urinary protein levels, e.g., to identify elevated levels, and determining whether the subject is at risk of death or heart failure hospital admission (DHFA) based on the analysis, and may further include treating the subject based on the risk determination.

### CROSS-REFERENCE TO RELATED APPLICATIONS

The present application claims priority to and benefit of U.S. Provisional Patent Application Nos. 63/485,770 and 63/488,158, filed Feb. 17, 2023 and Mar. 2, 2023, respectively, the entire contents of each of which are incorporated herein by reference.

### TECHNICAL FIELD

The present invention relates to methods of prognosis and treatment of subjects with heart failure with preserved ejection fraction based on urinary protein levels.

### BACKGROUND

Heart failure (HF) with preserved ejection fraction (HFpEF) represents approximately half of all HF diagnoses. There are no medical therapies that have been established to reduce all-cause mortality in HFpEF and therapeutic options to reduce the risk of cardiovascular death or hospitalization are limited. There is a need for prognostic methods in HFpEF to identify subjects at risk of cardiovascular death or hospitalization. Such prognosis may allow for medical and therapeutic intervention to reduce the risk of death or hospitalization.

Urinary proteins/peptides (UPPs) are noninvasive, inexpensive to collect, and usually without side effects or complications. Of note, the urinary proteome contains information not only from the kidney and the urinary tract but also from other organs via glomerular filtration of some plasma proteins. Urinary protein/peptides have potential as disease biomarkers.

### SUMMARY

Methods of prognosis and treatment of subjects with heart failure with preserved ejection fraction (HFpEF) based on urinary protein levels are described herein. Such methods may include obtaining a urine sample of a subject (e.g., a HFpEF patient), measuring urinary protein levels in the sample, analyzing the urinary protein levels, e.g., to identify elevated levels, and determining whether the subject is at risk of death or heart failure hospital admission (DHFA) based on the analysis, and may further include treating the subject based on the risk determination.

### DRAWINGS

FIGS. 1A-1B are volcano plots showing standardized hazard ratios for urinary proteins associated with the composite outcome of death or heart failure hospital admissions (DHFA) in non-adjusted analyses (1A) and after adjustment for urinary creatinine and the MAGGIC score (1B). The dashed lines represent the uncorrected (lower) and corrected (upper) significance level.

### DETAILED DESCRIPTION

As described above, there is a need for prognostic methods in HFpEF for risk stratification, including identifying subjects at risk of death or heart failure hospital admission (DHFA), and to treat a subject based on their prognosis. Previous proteomics studies in HFpEF have focused on proteins in blood plasma. Research regarding urinary proteomics in HFpEF is scarce (see, e.g., He T, et al. European journal of heart failure. 2021; 23 (11): 1875-87) and no data was previously available regarding the prognostic value of urinary proteomics in HFpEF.

It has presently been discovered that certain urinary proteins have prognostic value in HFpEF risk stratification. For example, certain urinary proteins can be used to determine risk of DHFA. The present disclosure provides prognostic methods for determining the severity of HFpEF, including determining the risk of DHFA, in a subject by measuring and/or analyzing the levels of one or more urinary proteins in a subject. These and other prognostic methods and various embodiments thereof are described in further detail below.

Prognosis and risk stratification based on the urinary protein levels can be applied in methods of treating a subject, e.g., a HFpEF patient. Prognosis may be determined prior to treatment, following treatment, and/or periodically during treatment, and the prognosis and/or change in prognosis, may be used to determine the course of treatment. For example, a subject at high risk for DHFA may receive a more aggressive treatment or a different therapeutic than a subject at low risk for DHFA. These and other methods of treatment and various embodiments thereof are described in further detail below.

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

Unless defined otherwise, technical and scientific terms used herein have the same meaning as commonly understood by a person of ordinary skill in the art. Any methods, devices and materials similar or equivalent to those described herein can be used in the practice of this invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The terms “a” or “an,” as used in herein means one or more.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

As used herein, “treatment” or “treating,” or “ameliorating” are used interchangeably herein. These terms refer to an approach for obtaining beneficial or desired results including but not limited to a therapeutic benefit. Therapeutic benefit means eradication or amelioration of the underlying disorder being treated and/or eradication or amelioration of one or more of the physiological symptoms associated with the underlying disorder such that an improvement is observed in the subject, notwithstanding that the subject may still be afflicted with the underlying disorder. Treatment includes causing the clinical symptoms of the disease to slow in development by administration of a composition; suppressing the disease, that is, causing a reduction in the clinical symptoms of the disease; inhibiting the disease, that is, arresting the development of clinical symptoms by administration of a composition after the initial appearance of symptoms; and/or relieving the disease, that is, causing the regression of clinical symptoms by administration of a composition after their initial appearance.

“Patient” or “subject” refers to a living organism suffering from or prone to a disease or condition that can be treated by using the methods provided herein. The term does not necessarily indicate that the subject has been diagnosed with a particular disease, but typically refers to an individual under medical supervision. Non-limiting examples include humans, other mammals, bovines, rats, mice, dogs, cats, monkeys, goat, sheep, cows, deer, and other non-mammalian animals. In some embodiments, a patient or subject is a human. In some embodiments, the patient or subject is suffering from or has been diagnosed with HFpEF.

### EMBODIMENTS

The present disclosure provides prognostic methods for HFpEF based on urinary protein levels. In one aspect, provided herein is a method for determining a risk of death or heart failure hospital admission (DHFA) in a subject comprising: measuring, or having measured, one or more urinary protein levels in the subject's urine; analyzing, or having analyzed, the one or more urinary protein levels; and determining the risk of DHFA in the subject based on the analysis of the one or more urinary protein levels.

The present disclosure also provides methods of treating HFpEF in conjunction with a subject's prognosis. In one aspect, provided herein is a method of treating heart failure with preserved ejection fraction (HFpEF) in a subject in need thereof, comprising: measuring, or having measured, one or more urinary protein levels in the subject's urine; analyzing, or having analyzed, the one or more urinary protein levels; determining, or having determined, the risk of death or heart failure hospital admission (DHFA) in the subject based on the analysis of the one or more urinary protein levels; and treating the subject with a HFpEF therapy. In some embodiments, if the subject is determined to be at risk of DHFA, then a first HFpEF therapy is administered, and if the subject is determined to not be at risk of DHFA, then a second HFpEF therapy is administered. In some embodiments, if the subject is determined to be at high risk of DHFA, then a first HFpEF therapy is administered, and if the subject is determined to be at low risk of DHFA, then a second HFpEF therapy is administered. In some embodiments, the first HFpEF therapy and the second HFpEF therapy each comprise administration of the same therapeutic, and the first HFpEF therapy comprises administration of a higher dose of the therapeutic compared to the second HFpEF therapy. In some embodiments, the first HFpEF therapy comprises administration of a therapeutic that is different from the second HFpEF therapy.

In some embodiments, the first HFpEF therapy and/or second HFpEF therapy is a guidline-directed medical therapy (GDMT) for HFpEF or associated comorbidities, such as hypertension and coronary artery disease. In some embodiments, the first HFpEF therapy and/or second HFpEF therapy is selected from the group consisting of a beta blocker, an angiotensin-converting enzyme (ACE) inhibitor, an angiotensin receptor blocker (ARB), an angiotensin receptor-neprilysin (ARN) inhibitor, a mineralocorticoid receptor antagonist (MRA), a SGLT2 inhibitor, a diuretic, a GLP-1 agonist, a calcium sensitizer, a soluble guanylate cyclase stimulator, and ivabradine, or any combination thereof. In some embodiments, the beta blocker is selected from the group consisting of bisoprolol, carvedilol, carvedilol, and metoprolol succinate, including controlled release and extended release versions thereof and pharmaceutically acceptable salts thereof. In some embodiments, the ACE inhibitor is selected from the group consisting of captopril, enalapril, fosinopril, lisinopril, perindopril, quinapril, ramipril, and trandolapril, and pharmaceutically acceptable salts thereof. In some embodiments, the ARB is selected from the group consisting of candesartan, losartan, and valsartan, and pharmaceutically acceptable salts thereof. In some embodiments, the ARN inhibitor is sacubitril-valsartan. In some embodiments, the MRA is spironolactone or eplerenone, or pharmaceutically acceptable salts thereof. In some embodiments, the soluble guanylate cyclase stimulator is vericiguat or digoxin, or pharmaceutically acceptable salts thereof. In some embodiments, the diuretic is selected from the group consisting of bumetanide, furosemide, torsemide, chlorthiazide, chlorthalidone, hydrochlorothiazide, indapamide, and metolazone, or a pharmaceutically acceptable salt thereof. In some embodiments, the diuretic is selected from the group consisting of bumetanide, furosemide, and torsemide, or a pharmaceutically acceptable salt thereof. In some embodiments, the GLP-1 agonist is selected from the group consisting of tirzepatide, semaglutide, lixisenatide, liraglutide, exenatide, and dulaglutide, or a pharmaceutically acceptable salt thereof. In some embodiments, the GLP-1 agonist is tirzepatide or semaglutide, or a pharmaceutically acceptable salt thereof. In some embodiments, the calcium sensitizer is levosimendan or a pharmaceutically acceptable salt thereof.

In some embodiments, the second HFpEF therapy is selected from the group consisting of a beta blocker, an angiotensin-converting enzyme (ACE) inhibitor, an angiotensin receptor blocker (ARB), an angiotensin receptor-neprilysin (ARN) inhibitor, and a mineralocorticoid receptor antagonist (MRA). In some embodiments, the second HFpEF therapy is selected from the group consisting of a beta blocker, an angiotensin receptor blocker (ARB), and a mineralocorticoid receptor antagonist (MRA). In some embodiments, the second HFpEF therapy is a beta blocker.

In some embodiments, the first HFpEF therapy is (a) a different guideline-directed medical therapy for HFpEF than the second HFpEF therapy or (b) a different dose (e.g., higher dose) of the same guideline-directed medical therapy as the second HFpEF therapy. In some embodiments, the first HFpEF therapy may be selected from the group consisting of a beta blocker, an angiotensin-converting enzyme (ACE) inhibitor, an angiotensin receptor blocker (ARB), an angiotensin receptor-neprilysin (ARN) inhibitor, a mineralocorticoid receptor antagonist (MRA), a SGLT2 inhibitor, a diuretic, a GLP-1 agonist, a calcium sensitizer, a soluble guanylate cyclase stimulator, and ivabradine. In some embodiments, the first HFpEF therapy is a more aggressive therapy. In some embodiments, the first HFpEF therapy is a higher dose of the second HFpEF therapy. In some embodiments, the first HFpEF therapy is a SGLT2 inhibitor. In some embodiments, the SGLT2 inhibitor is dapagliflozin or a pharmaceutically acceptable salt thereof. In some embodiments, the SGLT2 inhibitor is empagliflozin or a pharmaceutically acceptable salt thereof. In some embodiments, the first HFpEF therapy is aficamten or a pharmaceutically acceptable salt thereof.

In some embodiments, the first HFpEF therapy includes administration of multiple therapeutic agents, e.g., as described above. For example, the first HFpEF therapy may include a beta blocker and an SGLT2 inhibitor. The first HFpEF therapy may include a beta blocker and a ARB, a beta blocker and a MRA, a ARB and a MRA, or all three of a beta blocker, a ARB and a MRA.

In some embodiments, the first HFpEF therapy may include administration of a dose of a therapeutic (e.g., as described above) that is greater than the corresponding dose of the therapeutic according to the second HFpEF therapy. For example, the first HFpEF therapy dose may be 1.5× or more, 2× or more, 3× or more, or 4× or more the dose according to the second HFpEF therapy. For example, the first HFpEF therapy dose may be 1.5 to 2 times, 1.5 to 3 times, 2 to 3 times, 2 to 4 times, 3 to 5 times or 4 to 5 times more than the dose according to the second HFpEF therapy.

More generally, the urinary protein levels can be used for prognosis and risk stratification in HFpEF. In another aspect, provided herein is a method for assessing the severity of HFpEF in a subject comprising: measuring, or having measured, one or more urinary protein levels in the subject's urine; analyzing, or having analyzed, the one or more urinary protein levels; and determining the severity of HFpEF in the subject based on the analysis of the one or more urinary protein levels. In some embodiments, the severity of HFpEF is characterized by the risk of death or heart failure hospital admission (DHFA) of the subject.

The present methods also include monitoring prognosis of the subject, e.g., to follow the subject's condition over time. Such monitoring may be useful to decide whether to maintain or alter the subject's course of treatment. In another aspect, provided herein is a method for monitoring the severity of HFpEF in a subject who is suffering from or has been diagnosed with HFpEF, comprising: measuring, or having measured, one or more urinary protein levels in the patient's urine; analyzing, or having analyzed, the one or more urinary protein levels; and determining the severity of HFpEF in the patient based on the analysis of the one or more urinary protein levels. In some embodiments, the severity of HFpEF is characterized by the risk of death or heart failure hospital admission (DHFA) of the subject.

It is envisioned that selection of certain drug(s) may be made based on the particular urinary protein(s) measured and analyzed, wherein said certain drug(s) are known to have a functional effect on the urinary protein(s). In another aspect, provided herein is a method for identifying therapeutic options for a subject at risk of death or heart failure hospital admission (DHFA), comprising:
(a) obtaining, or having obtained, a urinary sample from the subject;
(b) measuring, or having measured, one or more urinary protein levels in the sample;
(c) analyzing, or having analyzed, the urinary protein levels in the sample;
(d) selecting at least one urinary protein based on the analysis; and
(e) selecting a drug or drug combination, wherein the drug or drug combination has a functional effect on the at least one urinary protein selected in (d).
   In some embodiments, the method further comprises (f) administering to the subject the drug or drug combination. In some embodiments, the method further comprises (g) measuring the at least one urinary protein selected in (d) following administration of the drug or drug combination, e.g., to determine if the urinary protein level has changed, such as to determine that the urinary protein level is no longer elevated.

The more general methods of risk stratification are applicable in treating HFpEF as well. In another aspect, provided herein is a method of treating heart failure with preserved ejection fraction (HFpEF) in a subject in need thereof, comprising: measuring, or having measured, one or more urinary protein levels in the subject's urine; analyzing, or having analyzed, the one or more urinary protein levels; determining, or having determined, the severity of HFpEF in the subject based on the analysis of the one or more urinary protein levels; and treating the subject with a HFpEF therapy.

In another aspect, provided herein is a method of determining a risk of death or heart failure hospital admission (DHFA) in a subject, comprising determining a risk of DHFA in the subject based on one or more urinary protein levels.

As mentioned above, more aggressive, less aggressive, or alternate therapies may be employed based on a subject's prognosis. In another aspect, provided herein is a method of treating heart failure with preserved ejection fraction (HFpEF) in a subject in need thereof, comprising: determining one or more urinary protein levels in the subject; and if the one or more urinary protein levels are above a threshold, then administering to the patient a first HFpEF therapy, and if the one or more urinary protein levels are below a threshold, then administering to the patient a second HFpEF therapy. In some embodiments, the second HFpEF therapy comprises a different drug than the first therapy. In some embodiments, the first HFpEF therapy comprises a first drug at a first dosage and the second therapy comprises the first drug at a second dosage.

In another aspect, provided herein is a method of treating heart failure with preserved ejection fraction (HFpEF) in a subject in need thereof, wherein the subject has been administered a first HFpEF therapy, comprising: determining one or more urinary protein levels in the subject; and if the one or more urinary protein levels are above a threshold, then administering to the patient a second HFpEF therapy. In some embodiments, the second HFpEF therapy comprises a different drug than the first therapy. In some embodiments, the first HFpEF therapy comprises a first drug at a first dosage and the second therapy comprises the first drug at a second dosage.

In another aspect, provided herein is a method of treating heart failure with preserved ejection fraction (HFpEF) in a subject in need thereof, wherein the subject has been administered a first HFpEF therapy, comprising: determining one or more urinary protein levels in the subject; and if the one or more urinary protein levels are below a threshold, then administering to the patient a second HFpEF therapy. In some embodiments, the second HFpEF therapy comprises a different drug than the first therapy. In some embodiments, the first HFpEF therapy comprises a first drug at a first dosage and the second therapy comprises the first drug at a second dosage.

In these and other aspects, the method may be practiced according to various embodiments, e.g., as described below.

In some embodiments, the subject is suffering from and/or has been diagnosed with heart failure with preserved ejection fraction (HFpEF). In some embodiments, the HFpEF is hypercontractile HFpEF. In some embodiments, the HFpEF is hypocontractile HFpEF. In some embodiments, the subject is suffering from and/or has been diagnosed with symptomatic heart failure. In some embodiments, the subject is suffering from and/or has been diagnosed with NYHA Class II, III, or IV heart failure. In some embodiments, the subject has symptomatic heart failure and a left ventricular ejection fraction (LVEF) greater than or equal to 45%. In some embodiments, the subject has symptomatic heart failure and a left ventricular ejection fraction (LVEF) greater than or equal to 50%.

In some embodiments of the present methods, the subject is determined to be at risk of HFpEF before the onset of HFpEF. In some embodiments, the subject is suffering from and/or has been diagnosed with hypertrophic cardiomyopathy (HCM) and is considered at risk for HFpEF. In some embodiments, the subject is suffering from and/or has been diagnosed with NYHA Class I heart failure and is considered at risk for HFpEF.

In some embodiments of the present methods, the subject is a HFpEF patient who is resistant to guideline-directed medical therapy or intolerant to guideline-directed medical therapy.

The present methods may include one or more of: measuring one or more urinary protein levels, analyzing the one or more urinary protein levels, and determining a prognosis of the subject (e.g., determining a risk of DHFA).

Analyzing the protein levels may include comparing the urinary protein levels to a threshold to determine whether the protein levels are elevated. Where multiple protein levels are measured, analyzing the protein levels may include calculating a composite score based on the protein levels, and comparing the composite score to a threshold. Where multiple protein levels are measured, analyzing protein levels may include analyzing whether a plurality, a majority, or a certain percentage (e.g., 60%, 65%, 70%, 75%, 80%, 85%, 90%, or 95%) of the protein levels are elevated. Analyzing the protein levels may also include determining the degree to which a protein level is elevated compared a normal or threshold level (e.g., a percentage above normal).

Determining a prognosis (e.g., risk of DHFA) may include referencing a correlation between the elevated level of the protein(s) and a prognosis (e.g., risk of DHA) to arrive at a prognosis for the subject. Determining a prognosis may further include delivering or outputting a prognosis of the subject.

Measuring one or more urinary protein levels may include measuring proteins in a urine sample by liquid chromatography-mass spectrometry (LC-MS). Other suitable methods known in the art for measuring urinary protein levels may be employed, such as other mass spectrometry methods and immunoassay methods. See e.g., Aitekenov S, et al., Review: Detection and quantification of proteins in human urine. Talanta. 2021 Feb. 1; 223 (Pt 1): 121718.

In some embodiments, the one or more urinary protein levels comprises the level of one or more proteins selected from the group consisting of MBD1, IGHA2, albumin, IGLV3-25, MAGEA4, KIF3A, APCS, Haptoglobin, COL15A1, COL6A1, ZG16B, ICOSLG, XPNPEP2, attractin, ALDH1A1, PGKA, PLAU, EGF, AMY2A, ATP1B1, SERPINA5, MELTF, HSP60, B4GALT1, CPE, AMY2B, SLC2A5, PROZ, DNASE1, ATP5F1A, MAN1A1, IGFALS, SLC12A3, prostasin, QPCT, HMCN1, C11orf54, LRRC19, ANGPTL2, and X6R868 (including combinations thereof). Elevated levels of these 40 proteins were identified in the study of Example 1 as directly or inversely correlated with DHFA outcomes in HFpEF subjects in an unadjusted analysis. In some embodiments, the one or more urinary protein levels comprises the level of one or more proteins selected from the group consisting of DNASE1, AMY2A, ANGPTL2, EGF, ZG16B, ALDH1A1, ICOSLG, PLAU, HSP60, AMY2B, B4GALT1, SLC2A5, and COL15A1 (including combinations thereof).

In some embodiments, the one or more proteins includes Glutathione S-transferase Mu 3. Elevated levels of this protein were identified as correlated with DHFA outcomes in an analysis using a model that adjusted for urine creatinine as being correlated with DHFA outcomes in HFpEF subjects.

In some embodiments, the one or more urinary protein levels comprises the level of one or more proteins selected from the group consisting of MBD1, IGHA2, ANGPTL2, AMY2A, DNASE1, AMY2B, ALDH1A1, PLAU, HSP60, ZG16B, ICOSLG, EGF, PGKA, C11orf54, ATP5F1A, SLC2A5, prostasin, ATP1B1, COL6A1, B4GALT1, and X6R868 (including combinations thereof). Elevated levels of these 21 proteins were identified in the study of Example 1 as directly or inversely correlated with DHFA outcomes in HFpEF subjects in a model that adjusted for urine creatinine levels and the Meta-Analysis Global Group in Chronic HF (MAGGIC) risk score. In some embodiments, the one or more urinary protein levels comprises the level of one or more proteins selected from the group consisting of ANGPTL2, AMY2A, DNASE1, ALDH1A1, AMY2B, PLAU, HSP60, ZG16B, ICOSLG, and PGKA (including combinations thereof). In some embodiments, the one or more urinary protein levels comprises the level of one or more proteins selected from the group consisting of ANGPTL2, AMY2A, DNASE1, ALDH1A1, and AMY2B (including combinations thereof).

In some embodiments, the urinary protein levels measured and/or analyzed are directly correlated with severity of HFpEF and/or increased risk of DHFA. In some embodiments, the one or more urinary protein levels comprises the level of one or more proteins selected from the group consisting of MBD1, IGHA2, albumin, IGLV3-25, MAGEA4, KIF3A, APCS, and Haptoglobin (including combinations thereof). These 8 proteins were found to be directly correlated with risk of DHFA in an unadjusted analysis. In some embodiments, the one or more urinary protein levels comprises the level of one or more proteins selected from the group consisting of MBD1 and IGHA2 (including combinations thereof). These 2 proteins were found to be directly correlated with risk of DHFA in a model that adjusted for urine creatinine levels and the Meta-Analysis Global Group in Chronic HF (MAGGIC) risk score.

In some embodiments, the urinary protein levels measured and/or analyzed are inversely correlated with severity of HFpEF and/or increased risk of DHFA. In some embodiments, the one or more urinary protein levels comprises the level of one or more proteins selected from the group consisting of COL15A1, COL6A1, ZG16B, ICOSLG, XPNPEP2, attractin, ALDH1A1, PGKA, PLAU, EGF, AMY2A, ATP1B1, SERPINA5, MELTF, HSP60, B4GALT1, CPE, AMY2B, SLC2A5, PROZ, DNASE1, ATP5F1A, MAN1A1, IGFALS, SLC12A3, prostasin, QPCT, HMCN1, C11orf54, LRRC19, ANGPTL2, and X6R868 (including combinations thereof). These 32 proteins were found to be inversely correlated with risk of DHFA in an unadjusted analysis. In some embodiments, the one or more urinary protein levels comprises the level of one or more proteins selected from the group consisting of ANGPTL2, AMY2A, DNASE1, AMY2B, ALDH1A1, PLAU, HSP60, ZG16B, ICOSLG, EGF, PGKA, C11orf54, ATP5F1A, SLC2A5, prostasin, ATP1B1, COL6A1, B4GALT1, and X6R868 (including combinations thereof). These 19 proteins were found to be inversely correlated with risk of DHFA in a model that adjusted for urine creatinine levels and the Meta-Analysis Global Group in Chronic HF (MAGGIC) risk score. In some embodiments, the one or more urinary protein levels comprises the level of one or more proteins selected from the group consisting of ANGPTL2, AMY2A and SLC2A5 (including combinations thereof).

In some embodiments, the urinary protein levels measured and/or analyzed are of proteins that are involved in fibrosis, such as collagen-derived proteins. For example, the urinary proteins may include COL15A1, COL6A1, or a combination thereof, which are collagen-derived proteins involved in fibrosis.

In some embodiments, the urinary protein levels measured and/or analyzed are of proteins that are involved in metabolism, such as carbohydrate metabolism or lipid metabolism. For example, the urinary proteins may include AMY2A, AMY2B, MAN1A1, or a combination thereof, which are related to carbohydrate metabolism. The urinary proteins may include ALDH1A1, B4GALT1, or a combination thereof, which are related to lipid metabolism. In some embodiments, the urinary protein levels measured and/or analyzed are of urinary proteins that are pancreatic alpha amylases, e.g., AMY2A, AMY2B, or a combination thereof.

In some embodiments, the urinary protein levels measured and/or analyzed are of proteins that are involved in inflammation. For example, the urinary proteins measured and/or analyzed may include ANGPTL2, which is known to have a role in chronic inflammation. The urinary proteins measured and/or analyzed may include HSPD1 and/or ATP1B1, which are involved in inflammation.

In some embodiments, the urinary protein levels measured and/or analyzed are of an epithelial sodium channel stimulator, such as prostasin.

In some embodiments, the urinary protein levels measured and/or analyzed includes the level of ANGPTL2.

In some embodiments, the urinary protein levels measured and/or analyzed includes the level of DNASE1.

In some embodiments, the urinary protein levels measured and/or analyzed includes the level of ALDH1A1.

In some embodiments, the urinary protein levels measured and/or analyzed includes the level of SLC2A5.

In some embodiments, the urinary protein levels measured and/or analyzed includes the level of AMY2A.

In some embodiments, the urinary protein levels measured and/or analyzed includes the level of AMY2B.

In some embodiments, the urinary protein levels measured and/or analyzed includes the level of MBD1.

In some embodiments, the urinary protein levels measured and/or analyzed includes the level of IGHA2.

In some embodiments, the urinary protein levels measured and/or analyzed includes the level of prostasin.

In some embodiments, analyzing the one or more urinary protein levels comprises determining whether the one or more urinary protein levels are elevated. In some embodiments, analyzing the one or more urinary protein levels comprises comparing the one or more urinary protein levels to a threshold. In some embodiments, determining the risk of DHFA comprises determining whether the one or more urinary protein levels are above or below a threshold.

In some embodiments, a method of the present disclosure further comprises determining that the subject is at increased risk of DHFA when the one or more urinary protein levels are elevated. In some embodiments, where increased risk of DHFA is correlated with elevated protein levels, the one or more urinary protein levels comprises the level of one or more proteins selected from the group consisting of MBD1, IGHA2, albumin, IGLV3-25, MAGEA4, KIF3A, APCS, and Haptoglobin (including combinations thereof). In some embodiments, where increased risk of DHFA is correlated with elevated protein levels, the one or more urinary protein levels comprises the level of one or more proteins selected from the group consisting of MBD1 and IGHA2 (including combinations thereof).

In some embodiments, a method of the present disclosure comprises determining that the subject is at decreased risk of DHFA when the one or more urinary protein levels are elevated. In some embodiments, where decreased risk of DHF A is correlated with elevated protein levels (or increased risk of DHFA is inversely correlated with elevated protein levels), the one or more urinary protein levels comprises the level of one or more proteins selected from the group consisting of COL15A1, COL6A1, ZG16B, ICOSLG, XPNPEP2, attractin, ALDH1A1, PGKA, PLAU, EGF, AMY2A, ATP1B1, SERPINA5, MELTF, HSP60, B4GALT1, CPE, AMY2B, SLC2A5, PROZ, DNASE1, ATP5F1A, MAN1A1, IGFALS, SLC12A3, prostasin, QPCT, HMCN1, C11orf54, LRRC19, ANGPTL2, and X6R868 (including combinations thereof). In some embodiments, where decreased risk of DHFA is correlated with elevated protein levels (or increased risk of DHFA is inversely correlated with elevated protein levels), the one or more urinary protein levels comprises the level of one or more proteins selected from the group consisting of ANGPTL2, AMY2A, DNASE1, AMY2B, ALDH1A1, PLAU, HSP60, ZG16B, ICOSLG, EGF, PGKA, C11orf54, ATP5F1A, SLC2A5, prostasin, ATP1B1, COL6A1, B4GALT1, and X6R868 (including combinations thereof). In some embodiments, where decreased risk of DHFA is correlated with elevated protein levels (or increased risk of DHFA is inversely correlated with elevated protein levels), the one or more urinary protein levels comprises the level of one or more proteins selected from the group consisting of ANGPTL2, AMY2A and SLC2A5 (including combinations thereof).

In some embodiments, a method of the present disclosure further comprises obtaining, or having obtained, a urine sample from the subject, from which the one or more urinary protein levels are measured. In some embodiments, the urine sample is frozen after being obtained and prior to measurement of urinary protein levels.

In some embodiments, a method of the present disclosure further comprises treating the subject with a therapy based on the subject's risk of DHFA. In some embodiments, the therapy is a HFpEF therapy.

In another aspect, provided herein is a method of treating heart failure with preserved ejection fraction (HFpEF) by administering a therapy to a subject in need thereof. In some embodiments, the therapy is a therapy known in the art for the treatment of HFpEF. In some embodiments, the subject is identified as in need of treatment based on the subject's levels of one or more urinary proteins. In some embodiments, the urinary proteins are one or more urinary proteins as disclosed herein, e.g., in Table 2. In some embodiments, the urinary protein(s) is MBD1 and/or IGHA2. In some embodiments, the urinary protein(s) is ANGPTL2, AMY2A and/or SLC2A5. In some embodiments, the method comprises one or more of: obtaining, or having obtained, a urine sample from the subject; measuring, or having measured, urinary protein levels in the sample; analyzing, or having analyzed, the urinary protein levels in the sample against a standard or threshold level; and determining, or having determined, whether to treat the subject based on the analysis. In some embodiments, the subject is monitored during treatment to determine need for treatment or adjustment of treatment. In some embodiments, the method comprises one or more of: obtaining, or having obtained, a urine sample from a subject receiving treatment for HFpEF; measuring, or having measured, urinary protein levels in the sample; analyzing, or having analyzed, the urinary protein levels in the sample against a standard or threshold level; and determining, or having determined, whether to stop, continue, or modify treatment of the subject based on the analysis. In some embodiments, the analysis comprises a quantitative analysis. In some embodiments, the analysis comprises correlating the measured urinary protein levels with the risk of DHFA. In some embodiments, the analysis of urinary protein(s) in a urine sample is used to determine a prognosis of the subject as being at risk (e.g., high risk) of death or heart failure hospital admission (DHFA). In some embodiments, the analysis or urinary protein(s) in a urine sample is used to determine a prognosis of the subject as being not at risk, or at low risk, of death or heart failure hospital admission (DHFA). In some embodiments, where the method includes a prognosis of the subject as at risk of DHFA (e.g., high risk), the method further includes stopping, continuing, or modifying treatment based on the prognosis, and/or the method further includes initiating other treatment or monitoring measures to reduce the risk of DHFA.

The present methods may include stopping, continuing or modifying treatment based on the urinary protein analysis (and/or related prognosis). The treatment stopped, continued, or modified may be a therapy (“T1”) selected from the group consisting of a beta blocker, an angiotensin-converting enzyme (ACE) inhibitor, an angiotensin receptor blocker (ARB), an angiotensin receptor-neprilysin (ARN) inhibitor, a mineralocorticoid receptor antagonist (MRA), a SGLT2 inhibitor, a diuretic, a GLP-1 agonist, a calcium sensitizer, a soluble guanylate cyclase stimulator, and ivabradine, including pharmaceutically acceptable salts thereof. For example, T1 may be stopped if it is determined that prognosis is worsening and/or urinary protein level(s) are not within a target range or at a target level (or are not progressing toward a target range or target level) during treatment with T1. T1 may be continued if it is determined that prognosis is improving and/or urinary protein level(s) are within a target range or at a target level (or are progressing toward a target range or target level) during treatment with T1. As another example, T1 may be modified if it is determined that prognosis is worsening or not improving during treatment with T1 and/or urinary protein level(s) are not within a target range or at a target level (or are not progressing toward a target range or target level).

Modifying T1 may include stopping T1 and initiating a second therapy (“T2”). Modifying T1 may include continuing T1 and initiating a second therapy (“T2”) together with T1. Modifying T1 may include increasing the dose of T1. Modifying T1 may include decreasing the dose of T1 with or without initiation of a second therapy (“T2”). In some embodiments, T2 is a more aggressive therapy than T1.

In some embodiments, T2 is an SGLT2 inhibitor. In some embodiments, T2 is aficamten or a pharmaceutically acceptable salt thereof. In some embodiments, T1 is a beta blocker, an angiotensin receptor blocker (ARB), or a mineralocorticoid receptor antagonist (MRA), and T2 is an SGLT2 inhibitor. In some embodiments, T1 is a beta blocker, an angiotensin receptor blocker (ARB), or a mineralocorticoid receptor antagonist (MRA), and T2 is aficamten or a pharmaceutically acceptable salt thereof.

In some embodiments, T2 is a less aggressive therapy than T1, e.g., a less aggressive drug. In some embodiments, modifying T1 comprises reducing the dose of the T1 therapy.

In some embodiments, T2 is therapy selected from the group consisting of a beta blocker, an angiotensin-converting enzyme (ACE) inhibitor, an angiotensin receptor blocker (ARB), an angiotensin receptor-neprilysin (ARN) inhibitor, a mineralocorticoid receptor antagonist (MRA), a SGLT2 inhibitor, a diuretic, a GLP-1 agonist, a calcium sensitizer, a soluble guanylate cyclase stimulator, and ivabradine, including pharmaceutically acceptable salts thereof, and which is different from T1.

In some embodiments, modifying T1 may include initiating a mechanical intervention with or without continued T1 therapy, and with or without initiation of a T2 therapy. In some embodiments, the mechanical intervention is mechanical circulatory support (MCS). In some embodiments, the mechanical intervention is an atrial shunt, a left ventricular expander, or a neurostimulator.

In some embodiments, T2 includes administration of multiple therapeutic agents, e.g., as described above. For example, T2 may include a SGLT2 inhibitor with one or more of a beta blocker, a ARB, and a MRA. T2 may include aficamten or a pharmaceutically acceptable salt thereof with one or more of a beta blocker, a ARB, and a MRA. T2 may include a beta blocker and a ARB, a beta blocker and a MRA, a ARB and a MRA, or all three of a beta blocker, a ARB and a MRA.

In some embodiments, T2 may include administration of a dose of a therapeutic (e.g., as described above) that is greater than the corresponding dose of the therapeutic according to T1. For example, the T2 dose may be 1.5× or more, 2× or more, 3× or more, or 4× or more than the dose according to the second HFpEF therapy. For example, the T2 dose may be 1.5 to 2 times, 1.5 to 3 times, 2 to 3 times, 2 to 4 times, 3 to 5 times or 4 to 5 times more than the dose according to T1.

In another aspect, provided herein is a method of diagnosing (or prognosing) a subject's risk of death or heart failure hospital admission (DHFA), the method comprising measuring urinary protein levels in the subject. In some embodiments, the subject is a HFpEF patient. In some embodiments, the HFpEF is hypercontractile HFpEF. In some embodiments, the HFpEF is hypocontractile HFpEF. In some embodiments, the urinary proteins are one or more urinary proteins as disclosed herein, e.g., in Table 2. In some embodiments, the urinary protein(s) is MBD1 and/or IGHA2. In some embodiments, the urinary protein(s) is ANGPTL2, AMY2A and SLC2A5. In some embodiments, the method comprises one or more of: obtaining, or having obtained, a urine sample from the subject, measuring, or having measured, urinary protein levels in the sample, analyzing, or having analyzed, the urinary protein levels in the sample against a standard or threshold level, and diagnosing (or prognosing), or having diagnosed (or prognosed), the subject's risk of DHFA based on the analysis. In some embodiments, the subject is determined to be at risk or high risk of DHFA when a urinary protein level is above a standard value, or above a threshold value. In some embodiments, the subject is determined to be at risk, or at high risk, of DHFA when the subject has elevated levels of a urinary protein selected from MBD1, IGHA2, or a combination thereof. In some embodiments, the subject is determined to be not at risk or at low risk of DHFA when a urinary protein level is above a standard value, or above a threshold value. In some embodiments, the subject is determined to be not at risk, or at low risk, of DHFA when the subject has elevated levels of a urinary protein selected from ANGPTL2, AMY2A and SLC2A5, or any combination thereof. In some embodiments, the method further comprises treating the subject with a therapy based on the subject's risk of DHFA.

Therapies for HFpEF are described in the art, such as in Heart Failure with Preserved Ejection Fraction: Mechanisms and Treatment Strategies, Kazunori Omote, Frederik H. Verbrugge, Barry A. Borlaug, Annual Review of Medicine 2022 73:1, 321-337, which is incorporated herein by reference in its entirety.

In another aspect, provided herein is a test kit for diagnosing (or prognosing) a subject's risk of DHFA or assessing the severity of HFpEF in a subject. In some embodiments, the kit includes a collection device for collecting and/or storing a urine sample. In some embodiments, the collection device may be a container, e.g., a sterile container. In some embodiments, the kit further comprises a measurement device for measuring, qualitatively or quantitatively, one or more urinary protein levels in the subject's urine. In some embodiments, the measurement device may be a urine protein test strip. In some embodiments, the kit further comprises software for analyzing the measured protein levels. The software may be configured for input of one or more urinary protein levels. The software may perform analysis comparing urinary protein level(s) to threshold(s). The analysis may identify the urinary protein level(s) as elevated or not elevated. The software may calculate a composite score, percentage of proteins at elevated levels, and/or degree to which a urinary protein level is elevated. The software may output a prognosis of the subject with regard to the severity of HFpEF. The software may output a determination of the risk of DHFA for the subject. In some embodiments, the software may output suggested therapeutic interventions based on the analysis of the measured protein levels.

### EXAMPLES

### Example 1—Urinary Proteomics and Outcomes in Heart Failure with Preserved Ejection Fraction

In this study, we performed de novo urinary protein/peptide measurements in frozen urine samples available from 426 participants enrolled in the Treatment of Preserved Cardiac Function HF with an Aldosterone Antagonist Trial (TOPCAT). We evaluated the relationship between urine biomarker levels and the risk of the death or HF-related hospital admission (DHFA) in this cohort.

The top urinary proteins/peptides associated with DHFA in the adjusted analyses included angiopoietin-like protein 2 (ANGPTL2) (HR=0.5731, CI=0.47-0.7, P=3.13E-05), alpha amylase 2A (AMY2A) (HR=0.5496, CI=0.44-0.69, P=0.0001), deoxyribonuclease-1 (DNASE1) (HR=0.5704, CI=0.46-0.71, P=0.0002), aldehyde dehydrogenase 1 family member A1 (ALDH1A1) (HR=0.5572, CI=0.47-0.72, P=0.0003), alpha amylase 2B (AMY2B) (HR=0.5841, CI=0.44-0.7, P=0.0003). Higher urinary levels of various proteins involved in fibrosis (collagen VI alpha-1, collagen XV alpha-1), metabolism (pancreatic alpha-amylase 2A/B, mannosidase alpha class 1A member 1) and inflammation (heat shock protein family D member 1, inducible T cell costimulatory ligand) were associated with a lower risk of DHFA.

### Methods

### Study Population

Individuals included in this analysis were participants of the Treatment of Preserved Cardiac Function HF with an Aldosterone Antagonist Trial (TOPCAT). TOPCAT was a multicenter, double-blinded, placebo-controlled randomized control trial of spironolactone that enrolled 3,445 adults with HFpEF (LVEF≥45%) from six countries from 2006-2012. The design, characteristics, inclusion/exclusion criteria, and results of the trial have been previously published. See Pitt B, Pfeffer M A, Assmann S F, Boineau R, Anand I S, Claggett B, et al. Spironolactone for heart failure with preserved ejection fraction. N Engl J Med. 2014; 370:1383-92.

### Urine Biomarker Samples

Urine samples for biomarker analyses were obtained from a subset of TOPCAT study participants who had available samples for de novo proteomic analyses. Approximately 270 μl of urine from participants were aliquoted into deep well 96 well plates. Each plate included 4 wells with a pooled normal healthy volunteer urine sample that were designated as quality control samples. Urine sample plates were stored at −80° C. until commencement of the experiment. Each plate was prepared for proteomics analysis on a separate day. Urinary proteins were subjected to reduction and alkylation by addition of 30 μl 0.1M DTT and 0.2M IAA, followed by incubation at 60° C. at 1000 rpm for 1 hr in a thermo-shaker. Samples were cooled to room temperature, then combined with 900 μl of cold acetonitrile and incubated overnight at −20° C. The plate was centrifuged at room temperature for 20 minutes at 2500 g in a plate-centrifuge. Supernatants were aspirated using a 1.2 ml pipette using multi-channel pipette. Protein pellets were washed by adding 1 ml of 100% acetonitrile at room temperature. The plate was shaken on a plate shaker for 5 minutes, and subsequently centrifuged for 10 minutes at 2500 g at room temperature. Finally, the supernatants were carefully aspirated leaving behind the clean pellet. The pellets were air dried for 5 minutes. Protein pellets were dissolved in 100 μl of freshly prepared 8M urea containing 100 mM tris HCL. Proteins were digested with 0.5 μg LysC (Wako chemicals; Richmond, VA) at 37° C. for 4 hours at 1000 rpm. The partially digested samples were diluted with 400 μl LCMS grade water and 1 μg of trypsin/LysC mix (Promega, Madison, WI) was added. The plate was incubated at 37° C. overnight at 1000 rpm to allow complete digestion of proteins. Peptide concentrations were measured using Tryptophan Fluorescence method.

### LC MS/MS Analysis

A total of 500 ng of each sample was loaded onto individual Evotips (Evosep; Denmark) and washed with 50 μl 0.1% formic acid (FA) followed by the addition of 100 μl storage solvent (0.1% FA) to keep the Evotips wet until analysis. The Evosep One system (Evosep) was coupled on-line to a QExactive HF mass spectrometer (Thermo Fisher Scientific; Waltham, MA) with a nanoelectrospray ion source (Thermo Fisher Scientific). Peptides were eluted from Evotips onto a Pepsep C-18 reversed phase column (ReproSil 3 μm, 120 Å, 8 cm 75 μm ID), and separated with a preset 30 sample/day gradient provided Evosep One system. MS data were acquired using Xcalibur software. A data-dependent method was used to dynamically choose the top 10 most abundant precursor ions from the survey scan using HCD fragmentation. Survey scan were acquired with a mass range of 400-1000 Th at a resolution of 60,000 at m/z 200. The maximum ion injection times for the survey scan and the MS/MS scans were 50 ms and 100 ms respectively, and the AGC target values were set to 3E6 and 1E5 respectively. The isolation window was set to 1.5 Th, and ions were fragmented with a normalized collision energy of 27. Unassigned precursor ion charge states, singly charged ions as well as ions of charge states above 8 were rejected. Peptide match was preferred, and dynamic exclusion was set to 40 seconds.

### Sample Bioinformatics Analysis

Mass spectra were analyzed using MaxQuant software version 1.6.6.0. The maximum allowed mass deviation was set to 4.5 ppm for monoisotopic precursor ions and 0.5 Da for MS/MS peaks. Enzyme specificity was set to Trypsin/P and a maximum of two missed cleavages were allowed. Carbamidomethylcysteine was set as a fixed modification, N-terminal acetylation and methionine oxidation as variable modifications. The spectra were searched against the human Uniprot sequence database combined with common contaminants and concatenated with the reversed versions of all sequences. Protein identification required at least one unique or razor peptide per protein group. Quantification in MaxQuant was performed using label free quantification (LFQ) algorithm with fast LFQ and a minimum ratio count of 1. The false positive rate was set to 1% at both peptide and protein level. Match between runs was selected with an alignment time window of 20 minutes and match time window of 0.7 minutes. Contaminants, reversed sequence identification and proteins only identified by site were excluded from further data analysis. Missing values were imputed with sample minimum LFQ intensities.

### IPA

Pathway enrichment analysis was performed using Ingenuity Pathway Analysis (IPA) software (Qiagen; Hilden, Germany; www.qiagen.com/ingenuity). See Krämer A, Green J, Pollard Jr J, Tugendreich S. Causal analysis approaches in ingenuity pathway analysis. Bioinformatics. 2014; 30 (4): 523-30. UPPs were identified according to their UniProt identification annotation and included in overrepresentation analyses if associated with the outcome at a nominal p-value threshold of 0.01. The analysis calculates a P value (Fisher exact test) quantifying the overlap, and a z score quantifying the likelihood and direction (upregulated or downregulated), between the proteomics pattern and known canonical pathways.

### Statistical Analyses

Characteristics of participants were assessed with mean and standard deviation (SD) for normally distributed variables and median and interquartile range (IQR) for non-normally distributed variables. We compared clinical characteristics between subjects who had urine proteomics data available and those who did not. We used the non-paired Student's t-test for continuous normally distributed variables, the Kruskal-Wallis test for non-normally distributed continuous variables, and the chi-squared or Fisher exact test for categorical variables, as appropriate.

The primary outcome for this analysis was the composite of death or HF admission (DHFA), as defined and used previously. See Chirinos J A, Zhao L, Jia Y, Frej C, Adamo L, Mann D, et al. Reduced Apolipoprotein M and Adverse Outcomes Across the Spectrum of Human Heart Failure. Circulation. 2020; 141 (18): 1463-76. We evaluated the relationship between urine biomarker levels and the risk of DHFA using Cox regression. We ran three different analyses to assess the impact of correcting for different clinical factors. These included: 1) models without adjustment, 2) models that adjusted for urine creatinine levels, and 3) models that adjusted for urine creatinine levels and the Meta-Analysis Global Group in Chronic HF (MAGGIC) risk score, which incorporates multiple demographic, clinical and laboratory parameters. See Pocock S J, Ariti C A, McMurray J J, Maggioni A, Kober L, Squire I B, et al. Predicting survival in heart failure: a risk score based on 39 372 patients from 30 studies. Eur Heart J. 2013; 34 (19): 1404-13. Statistical significance was defined as a 2-tailed P value <0.05. We corrected the alpha level for multiple comparisons based on the principal components underlying the variability of all measured UPPs. See Tromp J, Khan M A, Klip I T, Meyer S, de Boer R A, Jaarsma T, et al. Biomarker Profiles in Heart Failure Patients With Preserved and Reduced Ejection Fraction. J Am Heart Assoc. 2017; 6 (4). All probability values presented are 2-tailed. Analyses were performed using the MATLAB statistics and machine learning toolbox R2022a.

### Results

### TOPCAT Population

TOPCAT urine proteomics were performed via a low input high throughput workflow. A low volume of urine, 0.35 mL, was collected from a patient, having protein concentration from <50 μg/ml to >5000 μg/ml. A one-plate sample preparation method was followed using protein precipitation and enzymatic digestion. Peptide clean-up was performed using an iST-BCT kit with total peptide measurement with tryptophan fluorescence assay. Final measurement was performed using nanoLCBMS with low input (400 ng peptides/injection) and high throughput (1.5 days per plate). Unbiased relative protein quantification of >2300 proteins across the cohort.

A total of 426 (12.4%) subjects from TOPCAT had urinary protein data and were included in this analysis. The TOPCAT trial was a randomized, multi-center (233 sites, 6 countries), double-blind trial with 3445 patients with symptomatic heart failure (HF) & a left ventricular ejection fraction (LVEF) greater than or equal to 45%. Patients received placebo (n=1723) or spironolactone (n=1722, 15-45 mg daily).

A comparison of subjects with and without available urinary protein data is shown in Table 1. There was no statistically significant difference in age, race, glomerular filtration rate, or country between those with and without protein data. In general, individuals with urine proteomics data were more likely to be male and exhibited slightly higher body mass index (BMI). Those with urine proteomic data also tended to exhibit slightly higher prevalence of atrial fibrillation, a history of myocardial infarction, and hypertension, and were somewhat more likely to use angiotensin converting enzyme inhibitors/angiotensin II receptor blockers and statins. They also exhibited a significantly lower systolic blood pressure, although the quantitative difference was small (130 mmHg versus 128 mmHg in participants with vs. without urinary samples, respectively).

TABLE 1
Characteristics of TOPCAT participants
with and without urine proteomics data.

| | Participants without | | Participants with | | |
|---|---|---|---|---|---|
| | urine proteomic | | urine proteomic | | P |
| | samples (n = 3,016) | | samples (n = 426) | | value |
| Demographic | | | | | |
| characteristics | | | | | |
| Age, years | 69 | (61-76) | 69 | (61-77) | 0.2647 |
| Female sex | 1587 | (52.3) | 196 | (46) | 0.0147 |
| Race | | | | | 0.0551 |
| White | 2668 | (88.46) | 391 | (91.78) | |
| Black | 274 | (9.08) | 28 | (6.57) | |
| Asian | 16 | (0.53) | 3 | (0.70) | |
| Other | 67 | (2.22) | 3 | (0.70) | |
| Location | | | | | 0.2315 |
| Americas | 1535 | (50.90%) | 230 | (53.99%) | |
| Eastern Europe | 1481 | (49.10%) | 196 | (46.01%) | |
| Systolic BP, mmHg | 130 | (120-140) | 128 | (120-134) | <0.0001 |
| Diastolic BP, | 80 | (70-81) | 75 | (68-80) | <0.0001 |
| mmHg | | | | | |
| eGFR, ml/min/1.73 | 65.4 | (53.7-79.3) | 65.7 | (53.7-77.9) | 0.6747 |
| m² | | | | | |
| BMI (kg/m2) | 30.8 | (27.1-35.6) | 32 | (27.8-36.4) | 0.0150 |
| Medical history | | | | | |
| Myocardial | 757 | (25.11) | 136 | (31.92) | 0.0027 |
| infarction | | | | | |
| Stroke | 234 | (7.76) | 31 | (7.28) | 0.7257 |
| COPD | 349 | (11.58) | 54 | (12.68) | 0.5084 |
| Hypertension | 2742 | (90.95) | 404 | (94.84) | 0.0073 |
| Atrial fibrillation | 1033 | (34.26) | 180 | (42.25) | 0.0012 |
| Diabetes mellitus | 980 | (32.50) | 138 | (32.39) | 0.9639 |
| Medication use | | | | | |
| Beta blockers | 2334 | (77.41) | 342 | (80.82) | 0.1826 |
| Calcium channel | 1141 | (37.84) | 152 | (35.68) | 0.3881 |
| blocker | | | | | |
| ACE/ARB use | 2558 | (84.84) | 341 | (80.05) | 0.0110 |
| Aspirin use | 1972 | (65.41) | 278 | (65.26) | 0.9520 |
| Statin use | 1528 | (50.68) | 277 | (65.02) | <0.0001 |

Values represent median (interquartile range) or n (%). Abbreviations: eGFR=estimated glomerular filtration rate; ACE=angiotensin-converting enzyme, ARB=angiotensin receptor blocker, BMI=body mass index, COPD=chronic obstructive pulmonary disease; BP-blood pressure

### Association of UPP Levels and the Incidence of DHFA: Non-Adjusted Analyses

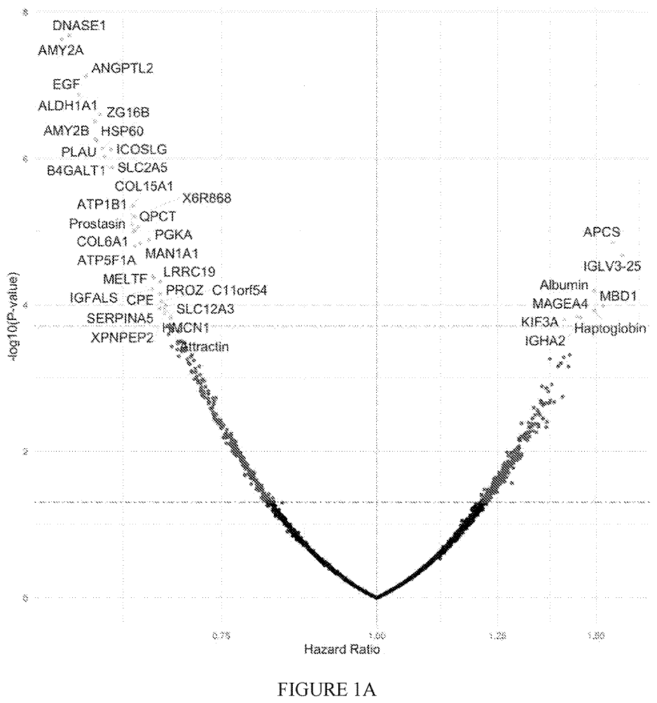

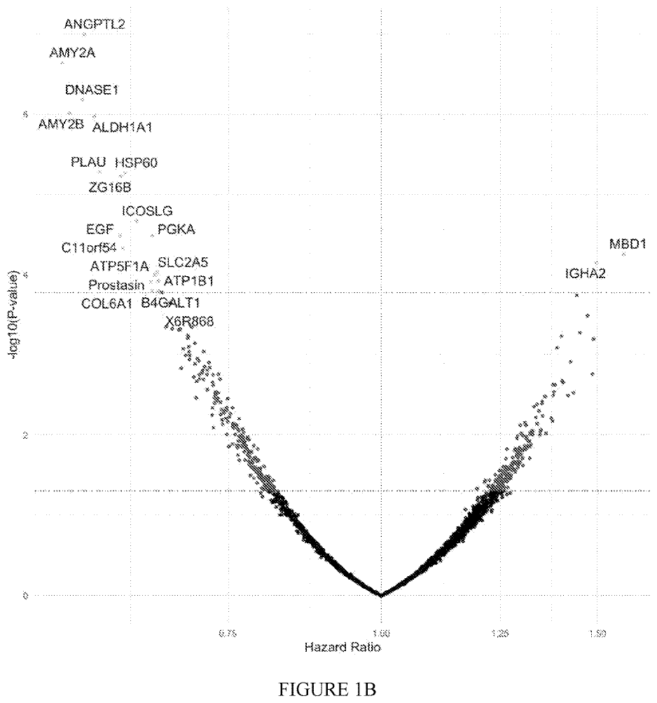

In non-adjusted analyses, we found 40 urinary proteins/peptides to be significantly associated with DHFA after alpha error correction. FIG. 1A shows a volcano plot representing the relationship between UPPs and the risk of DHFA. Table 2 lists UPPs that were significantly associated with DHFA, along with standardized HRs and 95% CIs. Table 3 lists the full list of name, function, and category of these proteins.

After alpha error correction for multiple comparisons, we found 40 urinary proteins significantly associated with DHFA in unadjusted analyses, 21 of which were also significant in adjusted analyses (FIG. 1B). Several identified proteins are involved in fibrosis, inflammation, renal sodium handling and metabolism.

TABLE 2
Urinary biomarkers associated with DHFA.

| | | NON-ADJUSTED | | | | ADJUSTED | | | |
|---|---|---|---|---|---|---|---|---|---|
| UNIPROT ID | Short name | Beta | HR | 95% CI | P-value | Beta | HR | 95% CI | P-value |
| A0A075B6N7 | IGHA2 | 0.38 | 1.458 | 1.2-1.77 | 0.045 | 0.40 | 1.4965 | 1.23-1.83 | 0.0218 |
| A0A087X0K0 | COL15A1 | −0.45 | 0.637 | 0.53-0.77 | 0.0014 | −0.40 | 0.6729 | 0.55-0.83 | 0.0677 |
| A0A087X0S5 | COL6A1 | −0.45 | 0.638 | 0.52-0.78 | 0.0031 | −0.43 | 0.6507 | 0.52-0.81 | 0.0486 |
| A0A0C4DGN4 | ZG16B | −0.51 | 0.594 | 0.49-0.73 | 0.0001 | −0.49 | 0.6137 | 0.5-0.76 | 0.0018 |
| J3KPF9 | KIF3A | 0.35 | 1.415 | 1.18-1.69 | 0.0476 | 0.33 | 1.3906 | 1.15-1.69 | 0.2265 |
| K4DIA0 | ICOSLG | −0.49 | 0.611 | 0.5-0.74 | 0.0002 | −0.46 | 0.6322 | 0.51-0.78 | 0.0067 |
| K7EPZ6 | MBD1 | 0.42 | 1.520 | 1.23-1.88 | 0.0317 | 0.45 | 1.5751 | 1.26-1.96 | 0.0172 |
| O43895 | XPNPEP2 | −0.39 | 0.675 | 0.55-0.83 | 0.04 | −0.28 | 0.7568 | 0.61-0.94 | 0.983 |
| O75882 | Attractin | −0.38 | 0.683 | 0.56-0.83 | 0.0455 | −0.37 | 0.6897 | 0.55-0.86 | 0.2313 |
| P00352 | ALDH1A1 | −0.52 | 0.599 | 0.49-0.73 | 0.0001 | −0.54 | 0.5572 | 0.47-0.72 | 0.0003 |
| P00558 | PGKA | −0.42 | 0.656 | 0.54-0.79 | 0.004 | −0.43 | 0.6507 | 0.53-0.8 | 0.0101 |
| P00738 | Haptoglobin | 0.4 | 1.495 | 1.22-1.83 | 0.0363 | 0.33 | 1.3859 | 1.12-1.71 | 0.5238 |
| P00749 | PLAU | −0.52 | 0.595 | 0.49-0.73 | 0.0002 | −0.53 | 0.5898 | 0.47-0.74 | 0.0016 |
| P01133 | EGF | −0.55 | 0.576 | 0.47-0.71 | 1.33e−7 | −0.49 | 0.6128 | 0.49-0.77 | 0.0102 |
| P01717 | IGLV3-25 | 0.45 | 1.576 | 1.28-1.94 | 0.0065 | 0.37 | 1.4509 | 1.18-1.79 | 0.1528 |
| P02768 | Albumin | 0.4 | 1.495 | 1.23-1.82 | 0.0196 | 0.39 | 1.4720 | 1.19-1.82 | 0.0967 |
| P02743 | APCS | 0.44 | 1.547 | 1.27-1.88 | 0.0044 | 0.37 | 14430 | 1.19-1.75 | 0.0552 |
| P04746 | AMY2A | −0.58 | 0.558 | 0.46-0.69 | 2.34e−8 | −0.6 | 0.5496 | 0.44-0.69 | 0.0001 |
| P05026 | ATP1B1 | −0.45 | 0.635 | 0.52-0.78 | 0.0025 | −0.42 | 0.6585 | 0.53-0.81 | 0.0367 |
| P05154 | SERPINA5 | −0.4 | 0.669 | 0.55-0.82 | 0.0341 | −0.3 | 0.7421 | 0.6-0.92 | 0.8695 |
| P08582 | MELTF | −0.41 | 0.660 | 0.54-0.81 | 0.0125 | −0.33 | 0.7164 | 0.58-0.89 | 0.4703 |
| P10809 | HSP60 | −0.51 | 0.602 | 0.49-0.74 | 0.0002 | −0.48 | 0.6188 | 0.5-0.76 | 0.0017 |
| P15291 | B4GALT1 | −0.5 | 0.604 | 0.49-0.74 | 0.0003 | −0.41 | 0.6607 | 0.53-0.82 | 0.0499 |
| P16870 | CPE | −0.4 | 0.670 | 0.55-0.82 | 0.0217 | −0.36 | 0.6974 | 0.56-0.87 | 0.3026 |
| P19961 | AMY2B | −0.52 | 0.594 | 0.48-0.73 | 0.0002 | −0.58 | 0.5841 | 0.44-0.7 | 0.0003 |
| P22732 | SLC2A5 | −0.49 | 0.612 | 0.5-0.75 | 0.0004 | −0.42 | 0.6574 | 0.53-0.81 | 0.0286 |
| P22891 | PROZ | −0.4 | 0.670 | 0.55-0.81 | 0.0149 | −0.31 | 0.7367 | 0.6-0.9 | 0.6072 |
| P24855 | DNASEI | −0.57 | 0.584 | 0.46-0.69 | 2.09e−8 | −0.56 | 0.5704 | 0.46-0.71 | 0.0002 |
| P25705 | ATP5F1A | −0.45 | 0.639 | 0.52-0.78 | 0.005 | −0.42 | 0.6539 | 0.53-0.81 | 0.0309 |
| P33908 | MAN1A1 | −0.44 | 0.645 | 0.53-0.79 | 0.0045 | −0.4 | 0.6681 | 0.53-0.84 | 0.1303 |
| P35858 | IGFALS | −0.42 | 0.659 | 0.54-0.81 | 0.0186 | −0.36 | 0.6983 | 0.56-0.87 | 0.2734 |
| P43358 | MAGEA4 | 0.37 | 1.448 | 1.2-1.75 | 0.0436 | 0.34 | 1.4019 | 1.16-1.7 | 0.1668 |
| P55017 | SLC12A3 | −0.39 | 0.677 | 0.56-0.82 | 0.0307 | 0.32 | 0.7297 | 0.6-0.89 | 0.5271 |
| Q16651 | Prostasin | −0.45 | 0.639 | 0.52-0.78 | 0.003 | −0.43 | 0.6493 | 0.52-0.81 | 0.0372 |
| Q16769 | QPCT | −0.44 | 0.643 | 0.53-0.78 | 0.0027 | −0.38 | 0.6842 | 0.55-0.85 | 0.1414 |
| Q96RW7 | HMCN1 | −0.39 | 0.675 | 0.55-0.82 | 0.0338 | −0.36 | 0.6970 | 0.56-0.86 | 0.2185 |
| Q9H0W9 | C11orf54 | −0.4 | 0.671 | 0.55-0.82 | 0.0274 | −0.48 | 0.6160 | 0.49-0.78 | 0.0146 |
| Q9H756 | LRRC19 | −0.41 | 0.663 | 0.54-0.81 | 0.0131 | −0.35 | 0.7038 | 0.56-0.88 | 0.4487 |
| Q9UKU9 | ANGPTL2 | −0.54 | 0.566 | 0.48-0.71 | 7.54e−8 | −0.56 | 0.5731 | 0.47-0.7 | 3.13E0−5 |
| X6R868 | X6R868 | −0.45 | 0.638 | 0.53-0.78 | 0.0019 | −0.42 | 0.6589 | 0.53-0.82 | 0.0488 |

HR = hazard ratio,
SE = standard error

Table 2 shows statistical association of urinary protein levels with the outcome either in unadjusted analyses (left) or analyses that adjusted for urine creatinine levels and the MAGGIC risk score (right).

TABLE 3
Name, function, and category of urinary proteins associated with
DHFA in our study in either non-adjusted or adjusted analyses.

| Short name | Full name | Function | Biologic process |
|---|---|---|---|
| IGHA2 | Immunoglobulin | Constant region of | Immune response |
| | heavy constant alpha | immunoglobulin heavy chains. | |
| | 2 (IGHA2) | | |
| COL15A1 | Collagen alpha-1(XV) | Collagen type XV is predominantly | Fibrosis |
| | chain | located in the basement membrane | |
| | | zones of microvessels and cardiac | |
| | | or skeletal myocytes | |
| COL6A1 | Collagen alpha-1(VI) | Collagen VI is a major structural | Fibrosis |
| | chain | component of microfibrils | |
| ZG16B | Zymogen granule | Carbohydrate binding activity, | Carbohydrate binding, |
| | protein 16 homolog B | retina homeostasis | retina homeostasis |
| Albumin | Albumin | Binds water, electrolytes, | |
| | | hormones, drugs. Regulates | |
| | | collodial osmotic pressure in blood. | |
| KIF3A | Kinesin-like protein | Microtubule-based anterograde | Microtubule-based |
| | | translocator for membranous | movement |
| | | organelles, cilia formation, | |
| | | centriole cohesion. | |
| ICOSLG | ICOS ligand | Ligand for T-cell specific receptor | Inflammatory response |
| | | ICOS | |
| MBD1 | Methyl-CpG-binding | Transcriptomal repressor | Cell replication, DNA |
| | domain protein 1 | | transcription |
| XPNPEP2 | Xaa-Pro | Metalloprotease which catalyzes | |
| | aminopeptidase 2 | the removal of a residue from the | |
| | | N-termini of peptides. May play a | |
| | | role in the metabolism of the | |
| | | vasodilator bradykinin. | |
| Attractin | Attractin | Involved in the initial immune | Inflammatory response |
| | | inflammatory response and may | |
| | | regulate chemokines. May play a | |
| | | role in melanocortin signaling | |
| | | pathways involved in energy | |
| | | homeostasis and hair color. | |
| ALDH1A1 | Aldehyde | Cytosolic dehydrogenase that | lipid metabolism |
| | dehydrogenase 1A1 | catalyzes oxidation of a wide range | |
| | | of aldehydes to carboxylic acids | |
| PGKA | Phosphoglycerate | Catalyzes one of the two ATP | Glycolysis |
| | kinase 1 | producing reactions in the | |
| | | glycolytic pathway | |
| Haptoglobin | Haptoglobin | Haptoglobin binds free | Acute phase response, |
| | | hemoglobin. It also has | immunity |
| | | antibacterial activity and plays a | |
| | | role in many aspects of the acute | |
| | | phase reactions. | |
| PLAU | Urokinase-type | Cleaves the zymogen plasminogen | Blood coagulation, |
| | plasminogen activator | to form the active enzyme plasmin. | fibrinolysis, |
| | | | hemostasis, |
| | | | plasminogen activation |
| EGF | Pro-epidermal growth | EGF stimulates the growth of | Many (negative |
| | factor | various epidermal and epithelial | regulation of |
| | | tissues | cholesterol efflux, |
| | | | endothelial cell |
| | | | migration, |
| | | | angiogenesis) |
| IGLV3-25 | Immunoglobulin | Region of the immunoglobulin | Adaptive immunity, |
| | lambda variable 3-25 | light chains that participates in the | immunity |
| | | antigen recognition | |
| APCS | Serum amyloid P- | Can interact with DNA and | Many (innate immune |
| | component | histones | response, complement |
| | | | activation, chaperone- |
| | | | mediated protein |
| | | | complex assembly, |
| | | | negative regulation of |
| | | | monocyte |
| | | | differentiation, protein |
| | | | folding) |
| AMY2A | Pancreatic alpha- | Hydrolyzes 1,4-alpha-glucosiside | Carbohydrate |
| | amylase | bonds in oligosaccharides and | metabolism |
| | | polysaccharides | |
| ATP1B1 | Sodium/potassium- | Na+/K+ ATPase is an important | Cell adhesion, |
| | transporting ATPase | membrane protein responsible for | immunity, innate |
| | subunit beta-1 | the electrochemical gradients of | immunity, ion |
| | | sodium and potassium ions. | transport, potassium |
| | | | transport |
| SERPINA5 | Plasma serine | Inactivates serine proteases. | Fertilization, lipid |
| | protease inhibitor | | transport, transport |
| MELTF | Melanotransferrin | Involved in cellular iron uptake. | iron transport, |
| | | Present on melanoma cells | coagulation, |
| | | | inflammation |
| HSPD1 | 60 kDa heat shock | Chaperonin associated with | Host-virus interaction |
| | protein, mitochondrial | mitochondrial protein import. May | |
| | | also prevent misfolding and | |
| | | promote proper assembly of | |
| | | polypeptides generated under stress | |
| | | conditions in the mitochondria | |
| B4GALT1 | Beta-1,4- | Membrane-bound glycoproteins | lipid metabolism |
| | galactosyltransferase | that transfer galactose to acceptor | |
| | 1 | sugars. | |
| CPE | Carboxypeptidase E | Involved in synthesis of | Many (cardiac LV |
| | | neuropeptides and peptide | morphogenesis, insulin |
| | | hormones. Catalyzes the release of | processing, |
| | | C-terminal arginine or lysine | neuropeptide signaling |
| | | residues. | pathway, peptide |
| | | | hormone secretion) |
| AMY2B | Alpha-amylase 2B | Hydrolyzes 1,4-alpha-glucosiside | Carbohydrate |
| | | bonds in oligosaccharides and | metabolism |
| | | polysaccharides | |
| SLC2A5 | Solute carrier family | Fructose transporter. Plays a role in | transport, sugar |
| | 2, facilitated glucose | the regulation of salt uptake. | transport |
| | transporter member 5 | | |
| PROZ | Vitamin K-dependent | Involved in hemostasis. Inhibits | Blood coagulation, |
| | protein Z | coagulation factor Xa. | hemostasis |
| DNASE1 | Deoxyribonuclease-1 | Serum endonuclease. Involved in | Apoptosis |
| | | cell death by apoptosis | |
| ATPSF1A | ATP synthase subunit | A subunit of mitochondrial ATP | ATP synthesis, |
| | alpha, mitochondrial | synthase. | hydrogen ion transport, |
| | | | ion transport, transport |
| MAN1A1 | Mannosyl- | Involved in the synthesis of | Carbohydrate |
| | oligosaccharide 1,2- | glycoproteins | metabolism, golgi |
| | alpha-mannosidase IA | | apparatus mannnose |
| | | | trimming, protein |
| | | | targeting to ER, |
| | | | ubiquitin-dependent |
| | | | ERAD pathway |
| IGFALS | Insulin-like growth | Growth promoting peptides. | Cell adhesion |
| | factor-binding protein | Increases the half-life of insulin | |
| | complex acid labile | like growth factors. | |
| | subunit | | |
| MAGEA4 | Melanoma-associated | Regulates cell proliferation | Negative regulation of |
| | antigen 4 | | apoptotic process, |
| | | | negative regulation of |
| | | | transcription by RNA |
| | | | polymerase II, positive |
| | | | regulation of cell cycle |
| SLC12A3 | Solute carrier family | Sodium and chloride ion | Ion transport, sodium |
| | 12 member 3 | cotransporter. Receptor for the | transport, symport, |
| | | cytokine IL18 | transport, potassium |
| | | | ion homeostasis |
| Prostasin | Prostasin | Trypsinogen, a serine protease. | Hydrolase, protease, |
| | | | serine protease |
| QPCT | Glutaminyl-peptide | A transferase. Involved in | Cellular protein |
| | cyclotransferase | neuroendocrine peptides. | modification process |
| HMCN1 | Hemicentin-1 | Involved in cytoskeleton | Cell cycle, cell |
| | | development, basement membrane | division, sensory |
| | | organization, and cell division. | transduction, vision |
| C11orf54 | Ester hydrolase | Enables hydrolase activity. | Hydrolase |
| | C11orf54 | | |
| LRRC19 | Leucine-rich repeat- | Involved in signaling pathways, | Host mediated |
| | containing protein 19 | inflammation. | regulation of intestinal |
| | | | microbiota |
| | | | composition, regulation |
| | | | of inflammatory |
| | | | response, toll-like |
| | | | receptor signaling |
| | | | pathway |
| ANGPTL2 | Angiopoietin-related | A member of the vascular | Multicellular organism |
| | protein 2 | endothelial growth factor family, | development |
| | | promotes inflammation | |
| X6R868 | Bile salt activated | Bile salt activated lipase | |
| | lipase | | |

Name, function, and category of proteins, as adapted from Uniprot and Genecards.

Of the 40 UPPs associated with DHFA, 8 were positively associated, whereas 32 were inversely associated with the risk of DHFA. Increasing levels of the following 8 proteins were associated with an increased risk of DHFA: Methyl-CpG-binding domain protein 1 (MBD1, K7EPZ6), Immunoglobulin heavy constant alpha 2 (IGHA2, A0A075B6N7), albumin (P02768), Immunoglobulin lambda variable 3-25 (IGLV3-25, P01717), Melanoma-associated antigen 4 (MAGEA4, P43358), Kinesin-like protein (KIF3A, J3KPF9), APCS (P02743) and Haptoglobin (P00738). For the following 32 proteins, increasing levels in the urine were associated with a lower risk of DHFA: COL15A1, COL6A1, ZG16B, ICOSLG, XPNPEP2, attractin, ALDH1A1, PGKA, PLAU, EGF, AMY2A, ATP1B1, SERPINA5, MELTF, HSP60, B4GALT1, CPE, AMY2B, SLC2A5, PROZ, DNASE1, ATP5F1A, MAN1A1, IGFALS, SLC12A3, prostasin, QPCT, HMCN1, C11orf54, LRRC19, ANGPTL2, and X6R868.

The top prognostic UPPs were deoxyribonuclease-1 (DNASE1) (HR=0.584, CI=0.46-0.69; P=2.09E-08), alpha amylase 2A (AMY2A) (HR=0.588, CI=0.460.69, P=2.34E-08), angiopoietin-like protein 2 (ANGPTL2) (HR=0.566, CI=0.48-0.71, P=7.54E-08), epidermal growth factor (EGF) (HR=0.576, CI=0.47-0.71, P=1.33E-07), zymogen granule protein 16B (ZG16B) (HR=0.594, CI=0.49-0.73, P=0.0001), aldehyde dehydrogenase 1 family member A1 (ALDH1A1) (HR=0.599, CI=0.49-0.73, P=0.0001), inducible T cell costimulatory ligand (ICOSLG) (HR=0.611, CI=0.5-0.74, P=0.0002), plasminogen activator urokinase (PLAU) (HR=0.595, CI=0.49-0.73, P=0.0002), heat shock protein 60 (HSP60) (HR=0.602, CI=0.49-0.74, P=0.0002), alpha amylase 2B (AMY2B) (HR=0.594, CI=0.48-0.73, P=0.0002), beta 1, 4 glactosyltransferase 1 (B4GALT1) (HR=0.604, CI=0.49-0.74, P=0.0003), solute carrier family 2 member 5 (SLC2A5) (HR=0.612, CI=0.5-0.75, P=0.0004), and Collagen alpha-1 (XV) chain (COL15A1) (HR=0.637, CI=0.53-0.77, P=0.0014).

UPPs positively associated with the risk of DHFA included immunoglobulin lambda variable 3-25 (IGLV3-25) (HR=1.576, CI=1.28-1.94, P=0.0065), serum amyloid P-component (APCS) (HR=1.547, CI=1.27-1.88, P=0.0044), methyl-CpG-binding domain protein 1 (MBD1) (HR=1.52, CI=1.23-1.88, P=0.0317), albumin (HR=1.495, CI=1.23-1.82, P=0.0196), haptoglobin (HR=1.495, CI=1.22-1.83, P=0.0363), immunoglobulin heavy constant alpha 2 (IGHA2) (HR=1.458, CI=1.2-1.77, P=0.045). All other UPPs were negatively associated with DHFA.

### Association of Urinary Biomarker Levels and the Incidence of DHFA: Adjusted Analyses

In models that adjusted for urine creatinine, we found 39 proteins significantly associated with the incidence of DHFA. All except for one (Glutathione S-transferase Mu 3; P21266) was also significantly associated with the outcome in the non-unadjusted analyses. Glutathione S-transferase Mu 3 was downregulated with Std. Beta-0.37, HR 0.6.

In models that adjusted for urinary creatinine and the MAGGIC score, we found 21 UPPs to be significantly associated with the risk of DHFA, all of which were significantly associated with this outcome in the non-adjusted analyses. The peptides/proteins correlated with DHFA (upregulated proteins) include MBD1 and IGHA2. Those inversely correlated with DHFA include ANGPTL2, AMY2A, DNASE1, AMY2B, ALDH1A1, PLAU, HSP60, ZG16B, ICOSLG, EGF, PGKA, C11orf54, ATP5F1A, SLC2A5, prostasin, ATP1B1, COL6A1, B4GALT1, and X6R868. Proteins ANGPTL2, AMY2A and SLC2A5 were identified as a subset of interest for measuring downregulated proteins.

The top UPPs associated with the risk of DHFA in these adjusted models included: angiopoietin-like protein 2 (ANGPTL2) (HR=0.5731, CI=0.47-0.7, P=3.13E-05), alpha amylase 2A (AMY2A) (HR=0.5496, CI=0.44-0.69, P=0.0001), deoxyribonuclease-1 (DNASE1) (HR=0.5704, CI=0.46-0.71, P=0.0002), aldehyde dehydrogenase 1 family member A1 (ALDH1A1) (HR=0.5572, CI=0.47-0.72, P=0.0003), alpha amylase 2B (AMY2B) (HR=0.5841, CI=0.44-0.7, P=0.0003), plasminogen activator urokinase (PLAU) (HR=0.5898, CI=0.47-0.74, P=0.0016), heat shock protein 60 (HSP60) (HR=0.6188, HR=0.5-0.76, P=0.0017), zymogen granule protein 16B (ZG16B) (HR=0.6137, CI=0.5-0.76, P=0.0018),), inducible T cell costimulatory ligand (ICOSLG) (HR=0.6322, CI=0.51-0.78, P=0.0067), and phosphoglycerate kinase 1 (PGKA) (HR=0.6507, CI=0.53-0.8, P=0.0101).

### IPA Pathway Analysis

IPA pathway overrepresentation analysis based on the UPPs associated with the risk of DHFA identified five canonical signaling pathways associated with the outcome. Four of these were fibrosis-related pathways (hepatic fibrosis/hepatic stellate cell activation, wound healing and idiopathic pulmonary fibrosis and GP6 signaling), whereas the remainder pathway (NAD signaling) is related to cell metabolism.

### DISCUSSION

We conducted a proteomic analysis of urinary biomarkers associated with the risk of DHFA in HFpEF. We identified 40 urinary proteins/peptides associated with this outcome, which are related to fibrosis, metabolism and inflammation. We identified 21 proteins that were associated with the risk of DHFA after adjustment for the MAGGIC risk score and urinary creatinine levels.

We found several UPPs found to be inversely associated with the risk of DHF A in HFpEF. Interestingly, higher serum levels of some of these proteins have been associated with poor outcomes in other populations. For example, whereas increased levels of ANGPTL2 in the urine were associated with decreased risk of DHFA in our study, previous literature has implicated ANGPTL2 as playing a causal role in cardiovascular disease and HF by its role in chronic inflammation. Increased levels of serum ANGPTL2 in a pathologically stressed heart accelerates cardiac dysfunction, while inhibiting ANGPTL2 can delay disease progression. We also found urokinase-type plasminogen activator (PLAU or suPAR) levels in urine to be inversely associated with the risk of DHFA. In previous studies, plasma levels of suPAR were associated with cardiovascular death or MI in patients with coronary artery disease (CAD) and in those with congestive HF (predominantly comprised of HFrEF patients).

Of note, plasma and urine levels of proteins generally exhibit poor correlations with each other. Whereas urine is produced predominantly as a result of plasma filtration in the kidney, the origin of proteins and peptides present in the urine is less clear.

Increased Levels of Fibrosis Peptides in Urine are Associated with a Decreased Risk of DHFA

We found increased urinary levels of two collagen-derived proteins COL15A1 (Collagen alpha-1 (XV) chain) and COL6A1 (Collagen alpha-1 (VI) chain) to be associated with a lower risk of DHFA. COL6A1 is a chain of type VI collagen, which is a major structural component of microfibrils. Interestingly, plasma levels of endotrophin, a peptide derived from the collagen VI alpha-3 chain, has been reported to be strongly and positively associated with the risk of adverse outcomes in HFpEF. COL15A1 is part of type XV collagen, which is widely expressed but frequently localized to the basement membrane. Derangements in collagen turnover and organization in cardiomyopathies are well established. They have also been specifically implicated as part of the pathophysiology of HFpEF. One study of urinary proteomics found elevated levels of urinary biomarkers associated with collagen metabolism, including COL6A1 and COL15A1, to be generally enriched in patients with both HFpEF and HFrEF. However, this study did not assess their association with outcomes. See He T, et al. Urinary peptides in heart failure: a link to molecular pathophysiology. European journal of heart failure. 2021; 23 (11): 1875-87.

Our findings regarding inverse associations between COL6A1 and COL15A1 and the risk of DHF A is counterintuitive, given the association between plasma biomarkers of tissue fibrosis and adverse outcomes in HFpEF. It should be noted that urinary protein/peptides have multiple determinants, and are not a direct representation of plasma levels. In addition to glomerular filtration, proteins can be secreted in the urinary tract by tubular cells and epithelial cells. For instance, it has been reported that the secretion of collagen by renal tubular and epithelial cells can be affected by exposure to albumin. In addition, there is variable reabsorption of filtered proteins by tubular cells, and there may be interindividual differences in the degradation of filtered or secreted proteins that may ultimately affect their measured levels in urine. Finally, it is possible that increased levels in urine could represent increased clearance of plasma collagen-derived peptides.

### Metabolic Pathways Implicated in Outcomes

We identified several UPPs that are related to metabolic processes, including carbohydrate metabolism (AMY2A pancreatic alpha-amylase, AMY2B alpha amylase 2B, MAN1A1 Mannosyl-oligosaccharide 1,2-alpha-mannosidase IA) and lipid metabolism (ALDH1A1 aldehyde dehydrogenase 1A1, B4GALT1 Beta-1,4-galactosyltransferase 1).

We found that increased urinary pancreatic alpha-amylase (AMY2A and AMY2B) protein is associated with a lower risk of DHFA. There is limited literature on the association of AMY2A and HF but one study found that levels of plasma AMY2A were lower in patients with HF compared to healthy controls. However, another study reported that plasma amylase levels are elevated in patients with severe but not mild HF. Of note, testing assays for urinary amylase are widely available, due to its role in the diagnosis of acute pancreatitis.

### Other New Associations

We found prostasin to be inversely associated with the risk of DHFA. Prostasin is an epithelial sodium channel stimulator. Decreased prostasin expression is associated with poor outcomes in colorectal cancer and oral squamous cell carcinoma. However, a recently study found that plasma prostasin levels are positively associated with diabetes risk and cancer mortality, whereas another study found that serum prostasin has an inverse association with physical activity in a population-based cohort. Whether the association between lower urinary prostasin levels and the increased risk of DHFA seen in our study is related to physical activity in these patients is unknown. To our knowledge prostasin has not been associated with HFpEF.

### Study Limitations

Our study should be interpreted in the context of its strengths and limitations. Strengths of our study include its well-characterized cohort, relatively long period of follow-up, prospectively adjudicated outcomes with a stringent criteria and methodology, and the unbiased nature of UPP measurements. Our study also has limitations. Urinary samples were not available from all TOPCAT participants. In addition, the inclusion of some participant with undiagnosed cardiac and/or renal amyloidosis cannot be excluded, which may confound the urinary proteome. Our study did not include an external validation cohort, given the limited availability of HFpEF cohorts with prospective follow-up and available urine samples. Finally, the origin of the UPPs measured in our study (contribution from circulating blood, secretion by the urinary tract, differential catabolism, etc.) could not be assessed.

### CONCLUSIONS

Our study reports the relationship between UPPs and the risk of DHFA in HFpEF. We identify several novel associations between UPPs and adverse outcomes in this patient population. Higher levels of urinary proteins involved in fibrosis, metabolism, and inflammation are associated with a lower risk of the composite outcome of DHFA in HFpEF. Many of the novel associations are independent of clinical risk scores and may aid in risk stratification in HFpEF.

We claim:

1. A method for determining a risk of death or heart failure hospital admission (DHFA) in a subject comprising: measuring, or having measured, one or more urinary protein levels in the subject's urine; analyzing, or having analyzed, the one or more urinary protein levels; and determining the risk of DHFA in the subject based on the analysis of the one or more urinary protein levels.

2. The method of claim 1, wherein the subject is suffering from and/or has been diagnosed with heart failure with preserved ejection fraction (HFpEF).

3. The method of claim 1, wherein the one or more urinary protein levels comprises the level of one or more proteins selected from the group consisting of MBD1, IGHA2, albumin, IGLV3-25, MAGEA4, KIF3A, APCS, Haptoglobin, COL15A1, COL6A1, ZG16B, ICOSLG, XPNPEP2, attractin, ALDH1A1, PGKA, PLAU, EGF, AMY2A, ATP1B1, SERPINA5, MELTF, HSP60, B4GALT1, CPE, AMY2B, SLC2A5, PROZ, DNASE1, ATP5F1A, MAN1A1, IGFALS, SLC12A3, prostasin, QPCT, HMCN1, C11orf54, LRRC19, ANGPTL2, and X6R868.

4. The method of claim 3, wherein the one or more urinary protein levels comprises the level of one or more proteins selected from the group consisting of MBD1, IGHA2, ANGPTL2, AMY2A, DNASE1, AMY2B, ALDH1A1, PLAU, HSP60, ZG16B, ICOSLG, EGF, PGKA, C11orf54, ATP5F1A, SLC2A5, prostasin, ATP1B1, COL6A1, B4GALT1, and X6R868.

5. The method of claim 1, wherein analyzing comprises determining whether the one or more urinary protein levels are elevated.

6. The method of claim 5, comprising determining that the subject is at increased risk of DHFA when the one or more urinary protein levels are elevated.

7. The method of claim 6, wherein the one or more urinary protein levels comprises the level of one or more proteins selected from the group consisting of MBD1, IGHA2, albumin, IGLV3-25, MAGEA4, KIF3A, APCS, and Haptoglobin.

8. The method of claim 7, wherein the one or more urinary protein levels comprises the level of one or more proteins selected from the group consisting of MBD1 and IGHA2.

9. The method of claim 5, comprising determining that the subject is at decreased risk of DHFA when the one or more urinary protein levels are elevated.

10. The method of claim 9, wherein the one or more urinary protein levels comprises the level of one or more proteins selected from the group consisting of COL15A1, COL6A1, ZG16B, ICOSLG, XPNPEP2, attractin, ALDH1A1, PGKA, PLAU, EGF, AMY2A, ATP1B1, SERPINA5, MELTF, HSP60, B4GALT1, CPE, AMY2B, SLC2A5, PROZ, DNASE1, ATP5F1A, MAN1A1, IGFALS, SLC12A3, prostasin, QPCT, HMCN1, C11orf54, LRRC19, ANGPTL2, and X6R868.

11. The method of claim 10, wherein the one or more urinary protein levels comprises the level of one or more proteins selected from the group consisting of ANGPTL2, AMY2A, DNASE1, AMY2B, ALDH1A1, PLAU, HSP60, ZG16B, ICOSLG, EGF, PGKA, C11orf54, ATP5F1A, SLC2A5, prostasin, ATP1B1, COL6A1, B4GALT1, and X6R868

12. The method of claim 11, wherein the one or more urinary protein levels comprises the level of one or more proteins selected from the group consisting of ANGPTL2, AMY2A and SLC2A5.

13. The method of any preceding claim, further comprising obtaining, or having obtained, a urine sample from the subject, from which the one or more urinary protein levels are measured.

14. The method of any preceding claim, wherein analyzing the one or more urinary protein levels comprises comparing the one or more urinary protein levels to a threshold.

15. The method of any preceding claim, wherein the HFpEF is hypercontractile HFpEF.

16. The method of any preceding claim, wherein the HFpEF is hypocontractile HFpEF.

17. The method of any preceding claim, further comprising treating the subject with a therapy based on the subject's risk of DHFA.

18. The method of claim 17, wherein the therapy is a HFpEF therapy.

19. The method of claim 18, wherein the therapy is selected from the group consisting of a beta blocker, an angiotensin-converting enzyme (ACE) inhibitor, an angiotensin receptor blocker (ARB), an angiotensin receptor-neprilysin (ARN) inhibitor, a mineralocorticoid receptor antagonist (MRA), a SGLT2 inhibitor, a diuretic, a GLP-1 agonist, a calcium sensitizer, a soluble guanylate cyclase stimulator, and ivabradine, or any combination thereof.

20. The method of any preceding claim, wherein the subject has symptomatic heart failure and a left ventricular ejection fraction (LVEF) greater than or equal to 45%.

21. The method of claim 13, wherein the urine sample is frozen after being obtained and prior to measurement of urinary protein levels.

22. The method of any preceding claim, wherein the one or more urinary protein levels in the subject's urine are measured using liquid chromatography-mass spectrometry (LC-MS).

23. The method of any preceding claim, wherein the one or more urinary protein levels comprises the level of one or more collagen derived proteins.

24. The method of claim 23, wherein the one or more collagen derived proteins comprises COL15A1 and/or COL6A1.

25. The method of any preceding claim, wherein the one or more urinary protein levels comprises the level of one or more pancreatic alpha amylases.

26. The method of claim 25, wherein the one or more pancreatic alpha amylases comprises AMY2A.

27. The method of claim 25, wherein the one or more pancreatic alpha amylases comprises AMY2B.

28. The method of any preceding claim, wherein the one or more urinary protein levels comprises the level of one or more urinary proteins selected from the group consisting of ANGPTL2, AMY2A, DNASE1, ALDH1A1, and AMY2B.

29. The method of any preceding claim, wherein the one or more urinary protein levels comprises the level of ANGPTL2.

30. The method of any preceding claim, wherein the one or more urinary protein levels comprises the level of DNASE1.

31. The method of any preceding claim, wherein the one or more urinary protein levels comprises the level of ALDH1A1.

32. The method of any preceding claim, wherein the one or more urinary protein levels comprises the level of SLC2A5.

33. The method of any preceding claim, wherein the one or more urinary protein levels comprises the level of prostasin.

34. The method of any preceding claim, wherein the one or more urinary protein levels comprises the level of one or more proteins selected from the group consisting of DNASE1, AMY2A, ANGPTL2, EGF, ZG16B, ALDH1A1, ICOSLG, PLAU, HSP60, AMY2B, B4GALT1, SLC2A5, and COL15A1.

35. The method of any preceding claim, wherein the one or more urinary protein levels comprises the level of one or more proteins selected from the group consisting of ANGPTL2, AMY2A, DNASE1, ALDH1A1, AMY2B, PLAU, HSP60, ZG16B, ICOSLG, and PGKA.

36. A method of treating heart failure with preserved ejection fraction (HFpEF) in a subject in need thereof, comprising:
measuring, or having measured, one or more urinary protein levels in the subject's urine;
analyzing, or having analyzed, the one or more urinary protein levels;
determining, or having determined, the risk of death or heart failure hospital admission (DHFA) in the subject based on the analysis of the one or more urinary protein levels; and
treating the subject with a HFpEF therapy.

37. The method of claim 36, wherein the one or more urinary protein levels comprises the level of one or more proteins selected from the group consisting of MBD1, IGHA2, albumin, IGLV3-25, MAGEA4, KIF3A, APCS, Haptoglobin, COL15A1, COL6A1, ZG16B, ICOSLG, XPNPEP2, attractin, ALDH1A1, PGKA, PLAU, EGF, AMY2A, ATP1B1, SERPINA5, MELTF, HSP60, B4GALT1, CPE, AMY2B, SLC2A5, PROZ, DNASE1, ATP5F1A, MAN1A1, IGFALS, SLC12A3, prostasin, QPCT, HMCN1, C11orf54, LRRC19, ANGPTL2, and X6R868.

38. The method of claim 37, wherein the one or more urinary protein levels comprises the level of one or more proteins selected from the group consisting of MBD1, IGHA2, ANGPTL2, AMY2A, DNASE1, AMY2B, ALDH1A1, PLAU, HSP60, ZG16B, ICOSLG, EGF, PGKA, C11orf54, ATP5F1A, SLC2A5, prostasin, ATP1B1, COL6A1, B4GALT1, and X6R868.

39. The method of claim 36, wherein analyzing comprises determining whether the one or more urinary protein levels are elevated.

40. The method of claim 39, comprising determining that the subject is at increased risk of DHFA when the one or more urinary protein levels are elevated.

41. The method of claim 40, wherein the one or more urinary protein levels comprises the level of one or more proteins selected from the group consisting of MBD1, IGHA2, albumin, IGLV3-25, MAGEA4, KIF3A, APCS, and Haptoglobin.

42. The method of claim 41, wherein the one or more urinary protein levels comprises the level of one or more proteins selected from the group consisting of MBD1 and IGHA2.

43. The method of claim 39, comprising determining that the subject is at decreased risk of DHFA when the one or more urinary protein levels are elevated.

44. The method of claim 43, wherein the one or more urinary protein levels comprises the level of one or more proteins selected from the group consisting of COL15A1, COL6A1, ZG16B, ICOSLG, XPNPEP2, attractin, ALDH1A1, PGKA, PLAU, EGF, AMY2A, ATP1B1, SERPINA5, MELTF, HSP60, B4GALT1, CPE, AMY2B, SLC2A5, PROZ, DNASE1, ATP5F1A, MAN1A1, IGFALS, SLC12A3, prostasin, QPCT, HMCN1, C11orf54, LRRC19, ANGPTL2, and X6R868.

45. The method of claim 44, wherein the one or more urinary protein levels comprises the level of one or more proteins selected from the group consisting of ANGPTL2, AMY2A, DNASE1, AMY2B, ALDH1A1, PLAU, HSP60, ZG16B, ICOSLG, EGF, PGKA, C11orf54, ATP5F1A, SLC2A5, prostasin, ATP1B1, COL6A1, B4GALT1, and X6R868.

46. The method of claim 45, wherein the one or more urinary protein levels comprises the level of one or more proteins selected from the group consisting of ANGPTL2, AMY2A and SLC2A5.

47. The method of any preceding claim, further comprising obtaining, or having obtained, a urine sample from the subject, from which the one or more urinary protein levels are measured.

48. The method of any preceding claim, wherein analyzing the one or more urinary protein levels comprises comparing the one or more urinary protein levels to a threshold.

49. The method of any preceding claim, wherein the HFpEF is hypercontractile HFpEF.

50. The method of any preceding claim, wherein the HFpEF is hypocontractile HFpEF.

51. The method of any preceding claim, wherein the HFpEF therapy is selected from the group consisting of a beta blocker, an angiotensin-converting enzyme (ACE) inhibitor, an angiotensin receptor blocker (ARB), an angiotensin receptor-neprilysin (ARN) inhibitor, a mineralocorticoid receptor antagonist (MRA), a SGLT2 inhibitor, a diuretic, a GLP-1 agonist, a calcium sensitizer, a soluble guanylate cyclase stimulator, and ivabradine, or any combination thereof.

52. The method of any preceding claim, further comprising, following treatment:
measuring, or having measured, one or more post-treatment urinary protein levels in the subject's urine;
analyzing, or having analyzed, the one or more post-treatment urinary protein levels; and
determining, or having determined, the post-treatment risk of DHFA in the subject based on the analysis of the one or more urinary protein levels.

53. The method of any preceding claim, wherein the subject has symptomatic heart failure and a left ventricular ejection fraction (LVEF) greater than or equal to 45%.

54. The method of claim 47, wherein the urine sample is frozen after being obtained and prior to measurement of urinary protein levels.

55. The method of any preceding claim, wherein the one or more urinary protein levels in the subject's urine are measured using liquid chromatography-mass spectrometry (LC-MS).

56. The method of any preceding claim, wherein the one or more urinary protein levels comprises the level of one or more collagen derived proteins.

57. The method of claim 56, wherein the one or more collagen derived proteins comprises COL15A1 and/or COL6A1.

58. The method of any preceding claim, wherein the one or more urinary protein levels comprises the level of one or more pancreatic alpha amylases.

59. The method of claim 58, wherein the one or more pancreatic alpha amylases comprises AMY2A.

60. The method of claim 58, wherein the one or more pancreatic alpha amylases comprises AMY2B.

61. The method of any preceding claim, wherein the one or more urinary protein levels comprises the level of one or more urinary proteins selected from the group consisting of ANGPTL2, AMY2A, DNASE1, ALDH1A1, and AMY2B.

62. The method of any preceding claim, wherein the one or more urinary protein levels comprises the level of ANGPTL2.

63. The method of any preceding claim, wherein the one or more urinary protein levels comprises the level of DNASE1.

64. The method of any preceding claim, wherein the one or more urinary protein levels comprises the level of ALDH1A1.

65. The method of any preceding claim, wherein the one or more urinary protein levels comprises the level of SLC2A5.

66. The method of any preceding claim, wherein the one or more urinary protein levels comprises the level of prostasin.

67. The method of any preceding claim, wherein the one or more urinary protein levels comprises the level of one or more proteins selected from the group consisting of DNASE1, AMY2A, ANGPTL2, EGF, ZG16B, ALDH1A1, ICOSLG, PLAU, HSP60, AMY2B, B4GALT1, SLC2A5, and COL15A1.

68. The method of any preceding claim, wherein the one or more urinary protein levels comprises the level of one or more proteins selected from the group consisting of ANGPTL2, AMY2A, DNASE1, ALDH1A1, AMY2B, PLAU, HSP60, ZG16B, ICOSLG, and PGKA.

69. The method of any preceding claim, wherein if the subject is determined to be at risk of DHFA, then administering a first HFpEF therapy, and if the subject is determined to not be at risk of DHFA, then administering a second HFpEF therapy.

70. The method of any preceding claim, wherein if the subject is determined to be at high risk of DHFA, then administering a first HFpEF therapy, and if the subject is determined to be at low risk of DHFA, then administering a second HFpEF therapy.

71. The method of claim 69 or 70, wherein the first HFpEF therapy and the second HFpEF therapy each comprise administration of the same therapeutic, and the first HFpEF therapy comprises administration of a higher dose of the therapeutic compared to the second HFpEF therapy.

72. The method of claim 69 or 70, wherein the first HFpEF therapy comprises administration of a therapeutic that is different from the second HFpEF therapy.

73. The method of any one of claims 69-72, wherein the first HFpEF therapy is selected from the group consisting of a beta blocker, an angiotensin-converting enzyme (ACE) inhibitor, an angiotensin receptor blocker (ARB), an angiotensin receptor-neprilysin (ARN) inhibitor, a mineralocorticoid receptor antagonist (MRA), a diuretic, a GLP-1 agonist, a calcium sensitizer, a soluble guanylate cyclase stimulator, and ivabradine, or a pharmaceutically acceptable salt thereof, or any combination thereof.

74. The method of any one of claims 69-72, wherein the first HFpEF therapy is a SGLT2 inhibitor.

75. The method of any one of claims 69-72, wherein the first HFpEF therapy is aficamten or a pharmaceutically acceptable salt thereof.

76. The method of any one of claims 69-75, wherein the second HFpEF therapy is selected from the group consisting of a beta blocker, an angiotensin-converting enzyme (ACE) inhibitor, an angiotensin receptor blocker (ARB), an angiotensin receptor-neprilysin (ARN) inhibitor, a mineralocorticoid receptor antagonist (MRA), a diuretic, a GLP-1 agonist, a calcium sensitizer, a soluble guanylate cyclase stimulator, and ivabradine, or any combination thereof.

77. A method for assessing the severity of HFpEF in a subject comprising:
measuring, or having measured, one or more urinary protein levels in the subject's urine;
analyzing, or having analyzed, the one or more urinary protein levels; and
determining the severity of HFpEF in the subject based on the analysis of the one or more urinary protein levels.

78. The method of claim 77, wherein the severity of HFpEF is characterized by the risk of death or heart failure hospital admission (DHFA) of the subject.

79. A method for monitoring the severity of HFpEF in a subject who is suffering from or has been diagnosed with HFpEF, comprising:
measuring, or having measured, one or more urinary protein levels in the subject's urine;
analyzing, or having analyzed, the one or more urinary protein levels; and
determining the severity of HFpEF in the subject based on the analysis of the one or more urinary protein levels.

80. The method of claim 79, wherein the severity of HFpEF is characterized by the risk of death or heart failure hospital admission (DHFA) of the subject.

81. A method for identifying therapeutic options for a subject at risk of death or heart failure hospital admission (DHFA), comprising:
(a) obtaining, or having obtained, a urinary sample from the subject;
(b) measuring, or having measured, one or more urinary protein levels in the sample;
(c) analyzing, or having analyzed, the urinary protein levels in the sample;
(d) selecting at least one urinary protein based on the analysis; and
(e) selecting a drug or drug combination, wherein the drug or drug combination has a functional effect on the at least one urinary protein selected in (d).

82. The method of claim 81, further comprising (f) administering to the subject the drug or drug combination.

83. The method of claim 82, further comprising (g) measuring the at least one urinary protein selected in (d) following administration of the drug or drug combination.

84. The method of any preceding claim, wherein the drug or drug combination is selected from the group consisting of a beta blocker, an angiotensin-converting enzyme (ACE) inhibitor, an angiotensin receptor blocker (ARB), an angiotensin receptor-neprilysin (ARN) inhibitor, a mineralocorticoid receptor antagonist (MRA), a diuretic, a GLP-1 agonist, a calcium sensitizer, a soluble guanylate cyclase stimulator, ivabradine, a SGLT2 inhibitor, and aficamten, or a pharmaceutically acceptable salt thereof, or any combination thereof.

85. A method of treating heart failure with preserved ejection fraction (HFpEF) in a subject in need thereof, comprising:
measuring, or having measured, one or more urinary protein levels in the subject's urine;
analyzing, or having analyzed, the one or more urinary protein levels;
determining, or having determined, the severity of HFpEF in the subject based on the analysis of the one or more urinary protein levels; and
treating the subject with a HFpEF therapy.

86. The method of any preceding claim, wherein the one or more urinary protein levels comprises the levels of one or more collagen derived proteins.

87. The method of any preceding claim, wherein the one or more collagen derived proteins comprises COL15A1 and/or COL6A1.

88. The method of any preceding claim, wherein the one or more urinary protein levels comprise the levels of one or more pancreatic alpha amylases.

89. The method of any preceding claim, wherein the one or more pancreatic alpha amylases comprises AMY2A.

90. The method of any preceding claim, wherein the one or more pancreatic alpha amylases comprises AMY2B.

91. The method of any preceding claim, wherein the one or more urinary protein levels comprise levels of one or more proteins selected from the group consisting of ANGPTL2, AMY2A, DNASE1, ALDH1A1, and AMY2B.

92. The method of any preceding claim, wherein the one or more urinary protein levels comprise the level of ANGPTL2.

93. The method of any preceding claim, wherein the one or more urinary protein levels comprises the level of DNASE1.

94. The method of any preceding claim, wherein the one or more urinary protein levels comprises the level of ALDH1A1.

95. The method of any preceding claim, wherein the one or more urinary protein levels comprises the level of MBD1 and/or IGHA2.

96. The method of any preceding claim, wherein the one or more urinary protein levels comprises the level of ANGPTL2, AMY2A, SLC2A5, or combinations thereof.

97. The method of any preceding claim, wherein the one or more urinary protein levels comprises the level of SLC2A5.

98. The method of any preceding claim, wherein the one or more urinary protein levels comprises the level of prostasin.

99. The method of any preceding claim, wherein the one or more urinary protein levels comprises the level of one or more proteins selected from the group consisting of MBD1, IGHA2, albumin, IGLV3-25, MAGEA4, KIF3A, APCS, Haptoglobin, COL15A1, COL6A1, ZG16B, ICOSLG, XPNPEP2, attractin, ALDH1A1, PGKA, PLAU, EGF, AMY2A, ATP1B1, SERPINA5, MELTF, HSP60, B4GALT1, CPE, AMY2B, SLC2A5, PROZ, DNASE1, ATP5F1A, MAN1A1, IGFALS, SLC12A3, prostasin, QPCT, HMCN1, C11orf54, LRRC19, ANGPTL2, and X6R868.

100. The method of any preceding claim, wherein the one or more urinary protein levels comprises the level of one or more proteins selected from the group consisting of MBD1, IGHA2, albumin, IGLV3-25, MAGEA4, KIF3A, APCS, and Haptoglobin.

101. The method of any preceding claim, wherein the one or more urinary protein levels comprises the level of one or more proteins selected from the group consisting of COL15A1, COL6A1, ZG16B, ICOSLG, XPNPEP2, attractin, ALDH1A1, PGKA, PLAU, EGF, AMY2A, ATP1B1, SERPINA5, MELTF, HSP60, B4GALT1, CPE, AMY2B, SLC2A5, PROZ, DNASE1, ATP5F1A, MAN1A1, IGFALS, SLC12A3, prostasin, QPCT, HMCN1, C11orf54, LRRC19, ANGPTL2, and X6R868.

102. The method of any preceding claim, wherein the one or more urinary protein levels comprises the level of one or more proteins selected from the group consisting of DNASE1, AMY2A, ANGPTL2, EGF, ZG16B, ALDH1A1, ICOSLG, PLAU, HSP60, AMY2B, B4GALT1, SLC2A5, and COL15A1.

103. The method of any preceding claim, wherein the one or more urinary protein levels comprises the level of one or more proteins selected from the group consisting of MBD1, IGHA2, ANGPTL2, AMY2A, DNASE1, AMY2B, ALDH1A1, PLAU, HSP60, ZG16B, ICOSLG, EGF, PGKA, C11orf54, ATP5F1A, SLC2A5, prostasin, ATP1B1, COL6A1, B4GALT1, and X6R868.

104. The method of any preceding claim, wherein the one or more urinary protein levels comprises the level of one or more proteins selected from the group consisting of ANGPTL2, AMY2A, DNASE1, ALDH1A1, AMY2B, PLAU, HSP60, ZG16B, ICOSLG, and PGKA.

105. The method of any preceding claim, wherein the HFpEF therapy is selected from the group consisting of a beta blocker, an angiotensin-converting enzyme (ACE) inhibitor, an angiotensin receptor blocker (ARB), an angiotensin receptor-neprilysin (ARN) inhibitor, a mineralocorticoid receptor antagonist (MRA), a diuretic, a GLP-1 agonist, a calcium sensitizer, a soluble guanylate cyclase stimulator, ivabradine, a SGLT2 inhibitor, and aficamten, or a pharmaceutically acceptable salt thereof, or any combination thereof.

106. A method of determining a risk of death or heart failure hospital admission (DHFA) in a subject, comprising determining a risk of DHFA in the subject based on one or more urinary protein levels.

107. The method of any preceding claim, further comprising measuring or having measured the one or more urinary protein levels in the subject's urine.

108. The method of any preceding claim, wherein the subject is suffering from and/or has been diagnosed with heart failure with preserved ejection fraction (HFpEF).

109. The method of any preceding claim, wherein the subject is suffering from and/or has been diagnosed with symptomatic heart failure.

110. The method of any preceding claim, wherein the subject is suffering from and/or has been diagnosed with NYHA Class II, III, or IV heart failure.

111. The method of any preceding claim, wherein determining the risk of DHFA comprises determining whether the one or more urinary protein levels are above or below a threshold.

112. The method of any preceding claim, wherein the one or more urinary protein levels comprises the levels of one or more collagen derived proteins.

113. The method of any preceding claim, wherein the one or more collagen derived proteins comprises COL15A1 and/or COL6A1.

114. The method of any preceding claim, wherein the one or more urinary protein levels comprise the levels of one or more pancreatic alpha amylases.

115. The method of any preceding claim, wherein the one or more pancreatic alpha amylases comprises AMY2A.

116. The method of any preceding claim, wherein the one or more pancreatic alpha amylases comprises AMY2B.

117. The method of any preceding claim, wherein the one or more urinary protein levels comprise levels of one or more proteins selected from the group consisting of ANGPTL2, AMY2A, DNASE1, ALDH1A1, and AMY2B.

118. The method of any preceding claim, wherein the one or more urinary protein levels comprise the level of ANGPTL2.

119. The method of any preceding claim, wherein the one or more urinary protein levels comprises the level of DNASE1.

120. The method of any preceding claim, wherein the one or more urinary protein levels comprises the level of ALDH1A1.

121. The method of any preceding claim, wherein the one or more urinary protein levels comprises the level of MBD1 and/or IGHA2.

122. The method of any preceding claim, wherein the one or more urinary protein levels comprises the level of ANGPTL2, AMY2A, SLC2A5, or combinations thereof.

123. The method of any preceding claim, wherein the one or more urinary protein levels comprises the level of SLC2A5.

124. The method of any preceding claim, wherein the one or more urinary protein levels comprises the level of prostasin.

125. The method of any preceding claim, wherein the one or more urinary protein levels comprises the level of one or more proteins selected from the group consisting of MBD1, IGHA2, albumin, IGLV3-25, MAGEA4, KIF3A, APCS, Haptoglobin, COL15A1, COL6A1, ZG16B, ICOSLG, XPNPEP2, attractin, ALDH1A1, PGKA, PLAU, EGF, AMY2A, ATP1B1, SERPINA5, MELTF, HSP60, B4GALT1, CPE, AMY2B, SLC2A5, PROZ, DNASE1, ATP5F1A, MAN1A1, IGFALS, SLC12A3, prostasin, QPCT, HMCN1, C11orf54, LRRC19, ANGPTL2, and X6R868.

126. The method of any preceding claim, wherein the one or more urinary protein levels comprises the level of one or more proteins selected from the group consisting of MBD1, IGHA2, albumin, IGLV3-25, MAGEA4, KIF3A, APCS, and Haptoglobin.

127. The method of any preceding claim, wherein the one or more urinary protein levels comprises the level of one or more proteins selected from the group consisting of COL15A1, COL6A1, ZG16B, ICOSLG, XPNPEP2, attractin, ALDH1A1, PGKA, PLAU, EGF, AMY2A, ATP1B1, SERPINA5, MELTF, HSP60, B4GALT1, CPE, AMY2B, SLC2A5, PROZ, DNASE1, ATP5F1A, MAN1A1, IGFALS, SLC12A3, prostasin, QPCT, HMCN1, C11orf54, LRRC19, ANGPTL2, and X6R868.

128. The method of any preceding claim, wherein the one or more urinary protein levels comprises the level of one or more proteins selected from the group consisting of DNASE1, AMY2A, ANGPTL2, EGF, ZG16B, ALDH1A1, ICOSLG, PLAU, HSP60, AMY2B, B4GALT1, SLC2A5, and COL15A1.

129. The method of any preceding claim, wherein the one or more urinary protein levels comprises the level of one or more proteins selected from the group consisting of MBD1, IGHA2, ANGPTL2, AMY2A, DNASE1, AMY2B, ALDH1A1, PLAU, HSP60, ZG16B, ICOSLG, EGF, PGKA, C11orf54, ATP5F1A, SLC2A5, prostasin, ATP1B1, COL6A1, B4GALT1, and X6R868.

130. The method of any preceding claim, wherein the one or more urinary protein levels comprises the level of one or more proteins selected from the group consisting of ANGPTL2, AMY2A, DNASE1, ALDH1A1, AMY2B, PLAU, HSP60, ZG16B, ICOSLG, and PGKA.

131. A method of treating heart failure with preserved ejection fraction (HFpEF) in a subject in need thereof, comprising:
determining one or more urinary protein levels in the subject; and
if the one or more urinary protein levels are above a threshold, then administering to the subject a first HFpEF therapy, and
if the one or more urinary protein levels are below a threshold, then administering to the subject a second HFpEF therapy.

132. A method of treating heart failure with preserved ejection fraction (HFpEF) in a subject in need thereof, wherein the subject has been administered a first HFpEF therapy, comprising:
determining one or more urinary protein levels in the subject; and
if the one or more urinary protein levels are above a threshold, then administering to the subject a second HFpEF therapy.

133. A method of treating heart failure with preserved ejection fraction (HFpEF) in a subject in need thereof, wherein the subject has been administered a first HFpEF therapy, comprising:
determining one or more urinary protein levels in the subject; and
if the one or more urinary protein levels are below a threshold, then administering to the subject a second HFpEF therapy.

134. The method of any preceding claim, wherein the second HFpEF therapy comprises a different drug than the first therapy.

135. The method of any preceding claim, wherein the first HFpEF therapy comprises a first drug at a first dosage and the second therapy comprises the first drug at a second dosage.

136. The method of any preceding claim, wherein the first HFpEF therapy is selected from the group consisting of a beta blocker, an angiotensin-converting enzyme (ACE) inhibitor, an angiotensin receptor blocker (ARB), an angiotensin receptor-neprilysin (ARN) inhibitor, a mineralocorticoid receptor antagonist (MRA), a diuretic, a GLP-1 agonist, a calcium sensitizer, a soluble guanylate cyclase stimulator, and ivabradine, or a pharmaceutically acceptable salt thereof, or any combination thereof.

137. The method of any preceding claim, wherein the first HFpEF therapy is a SGLT2 inhibitor.

138. The method of any preceding claim, wherein the first HFpEF therapy is aficamten or a pharmaceutically acceptable salt thereof.

139. The method of any preceding claim, wherein the second HFpEF therapy is selected from the group consisting of a beta blocker, an angiotensin-converting enzyme (ACE) inhibitor, an angiotensin receptor blocker (ARB), an angiotensin receptor-neprilysin (ARN) inhibitor, a mineralocorticoid receptor antagonist (MRA), a diuretic, a GLP-1 agonist, a calcium sensitizer, a soluble guanylate cyclase stimulator, and ivabradine, or a pharmaceutically acceptable salt thereof, or any combination thereof.

140. The method of any preceding claim, wherein the subject is suffering from and/or has been diagnosed with heart failure with preserved ejection fraction (HFpEF).

141. The method of any preceding claim, wherein the subject is suffering from and/or has been diagnosed with symptomatic heart failure.

142. The method of any preceding claim, wherein the subject is suffering from and/or has been diagnosed with NYHA Class II, III, or IV heart failure.

143. The method of any preceding claim, wherein the one or more urinary protein levels comprises the levels of one or more collagen derived proteins.

144. The method of any preceding claim, wherein the one or more collagen derived proteins comprises COL15A1 and/or COL6A1.

145. The method of any preceding claim, wherein the one or more urinary protein levels comprise the levels of one or more pancreatic alpha amylases.

146. The method of any preceding claim, wherein the one or more pancreatic alpha amylases comprises AMY2A.

147. The method of any preceding claim, wherein the one or more pancreatic alpha amylases comprises AMY2B.

148. The method of any preceding claim, wherein the one or more urinary protein levels comprise levels of one or more proteins selected from the group consisting of ANGPTL2, AMY2A, DNASE1, ALDH1A1, and AMY2B.

149. The method of any preceding claim, wherein the one or more urinary protein levels comprise the level of ANGPTL2.

150. The method of any preceding claim, wherein the one or more urinary protein levels comprises the level of DNASE1.

151. The method of any preceding claim, wherein the one or more urinary protein levels comprises the level of ALDH1A1.

152. The method of any preceding claim, wherein the one or more urinary protein levels comprises the level of MBD1 and/or IGHA2.

153. The method of any preceding claim, wherein the one or more urinary protein levels comprises the level of ANGPTL2, AMY2A, SLC2A5, or combinations thereof.

154. The method of any preceding claim, wherein the one or more urinary protein levels comprises the level of SLC2A5.

155. The method of any preceding claim, wherein the one or more urinary protein levels comprises the level of prostasin.

156. The method of any preceding claim, wherein the one or more urinary protein levels comprises the level of one or more proteins selected from the group consisting of MBD1, IGHA2, albumin, IGLV3-25, MAGEA4, KIF3A, APCS, Haptoglobin, COL15A1, COL6A1, ZG16B, ICOSLG, XPNPEP2, attractin, ALDH1A1, PGKA, PLAU, EGF, AMY2A, ATP1B1, SERPINA5, MELTF, HSP60, B4GALT1, CPE, AMY2B, SLC2A5, PROZ, DNASE1, ATP5F1A, MAN1A1, IGFALS, SLC12A3, prostasin, QPCT, HMCN1, C11orf54, LRRC19, ANGPTL2, and X6R868.

157. The method of any preceding claim, wherein the one or more urinary protein levels comprises the level of one or more proteins selected from the group consisting of MBD1, IGHA2, albumin, IGLV3-25, MAGEA4, KIF3A, APCS, and Haptoglobin.

158. The method of any preceding claim, wherein the one or more urinary protein levels comprises the level of one or more proteins selected from the group consisting of COL15A1, COL6A1, ZG16B, ICOSLG, XPNPEP2, attractin, ALDH1A1, PGKA, PLAU, EGF, AMY2A, ATP1B1, SERPINA5, MELTF, HSP60, B4GALT1, CPE, AMY2B, SLC2A5, PROZ, DNASE1, ATP5F1A, MAN1A1, IGFALS, SLC12A3, prostasin, QPCT, HMCN1, C11orf54, LRRC19, ANGPTL2, and X6R868.

159. The method of any preceding claim, wherein the one or more urinary protein levels comprises the level of one or more proteins selected from the group consisting of DNASE1, AMY2A, ANGPTL2, EGF, ZG16B, ALDH1A1, ICOSLG, PLAU, HSP60, AMY2B, B4GALT1, SLC2A5, and COL15A1.

160. The method of any preceding claim, wherein the one or more urinary protein levels comprises the level of one or more proteins selected from the group consisting of MBD1, IGHA2, ANGPTL2, AMY2A, DNASE1, AMY2B, ALDH1A1, PLAU, HSP60, ZG16B, ICOSLG, EGF, PGKA, C11orf54, ATP5F1A, SLC2A5, prostasin, ATP1B1, COL6A1, B4GALT1, and X6R868.

161. The method of any preceding claim, wherein the one or more urinary protein levels comprises the level of one or more proteins selected from the group consisting of ANGPTL2, AMY2A, DNASE1, ALDH1A1, AMY2B, PLAU, HSP60, ZG16B, ICOSLG, and PGKA.

162. The method of any preceding claim, wherein the subject is determined to be at risk of HFpEF before the onset of HFpEF.

163. The method of any preceding claim, wherein the subject is suffering from and/or has been diagnosed with hypertrophic cardiomyopathy (HCM) and is considered at risk for HFpEF.

164. The method of any preceding claim, wherein the subject is suffering from and/or has been diagnosed with NYHA Class I heart failure and is considered at risk for HFpEF.

165. The method of any preceding claim, wherein the subject is a HFpEF patient who is resistant to guideline-directed medical therapy.

166. The method of any preceding claim, wherein the subject is a HFpEF patient who is intolerant to guideline-directed medical therapy.
